(19) United States
(12) Patent Application Publication
(10) Pub. No.: US 2021/0388401 A1
(43) Pub. Date: Dec. 16, 2021

(54) METHODS OF REFINING A GRAIN OIL COMPOSITION, AND RELATED SYSTEMS, COMPOSITIONS AND USES

(71) Applicant: POET Research, Inc., Sioux Falls, SD (US)

(72) Inventors: Shannon S. Urban, Valley Springs, SD (US); Brett A. Flittie, Harrisburg, SD (US); Jacob A. Milbrandt, Tyndall, SD (US); Steven T. Bly, Sioux Falls, SD (US); Alexander T. McCurdy, Sioux Falls, SD (US)

(21) Appl. No.: 17/446,393

(22) Filed: Aug. 30, 2021

Related U.S. Application Data
(63) Continuation-in-part of application No. 16/775,822, filed on Jan. 29, 2020, now Pat. No. 11,104,922.
Application No. 16/775,822 is a continuation of application No. 15/988,794, filed on May 24, 2018, now Pat. No. 10,604,776.
(60) Provisional application No. 63/082,712, filed on Sep. 24, 2020.
(60) Provisional application No. 62/510,551, filed on May 24, 2017.

(51) Int. Cl. C12P 7/64; C12P 7/06; A23D 9/02; C11B 1/02; A23D 9/007; C11B 13/00; A23D 9/04; C11B 3/00; C12N 9/20

(52) U.S. Cl. CPC C12P 7/6454; C12P 7/06; A23D 9/02; C11B 1/025; A23D 9/007; C12N 2330/50; C12P 7/6436; A23D 9/04; C11B 3/003; C11B 3/008; C12N 9/20; C11B 13/00

(57) ABSTRACT

The present disclosure relates to methods and systems for refining grain oil compositions using an esterase enzyme component, water, bleaching processes, and combinations thereof, and related compositions produced therefrom having one or more reduced color values. The present disclosure also relates to methods of using said compositions, e.g., as mineral oil replacements.

### CROSS-REFERENCE TO RELATED APPLICATIONS

This application claims the benefit of U.S. Provisional Application No. 63/082,712, filed Sep. 24, 2020. This application is also a continuation-in-part of U.S. application Ser. No. 16/775,822 filed Jan. 29, 2020, which is a continuation of U.S. patent application Ser. No. 15/988,794, filed May 24, 2018, which claims the benefit of priority of U.S. Provisional Application No. 62/510,551, filed May 24, 2017. All of the above applications are incorporated herein by reference in their entirety.

### BACKGROUND

Ethanol biorefineries typically produce fuel-grade ethanol using a fermentation-based process. Much of the ethanol used for transportation fuel in the United States is produced from the fermentation of corn. In an exemplary dry-grind ethanol production process, a vegetable such as corn is delivered to a biorefinery and its particle size can be reduced by grinding the corn in a dry milling step. The resulting corn flour can then be combined with water, nutrients, enzymes, yeast, and/or other ingredients in a fermenter. Enzymes convert starch into fermentable sugars. Yeast converts fermentable sugars into ethanol. Fermentation results in a beer stream that includes, e.g., ethanol, water, suspended solids, dissolved solids, and corn oil. The beer stream is processed by a distillation unit where ethanol is removed. The stream from the distillation unit after ethanol has been recovered is referred to as whole stillage. This whole stillage stream includes, e.g., suspended solids, dissolved solids, water, and corn oil. The whole stillage stream is separated, typically by decanting centrifuges, into a thin stillage stream and a wet cake stream. The wet cake stream has a higher concentration of solids than whole stillage and is typically of a relatively high viscosity sludge-like consistency. The thin stillage has a lower concentration of suspended solids than whole stillage and is typically of a relatively low viscosity liquid stream.

The solids concentration of the thin stillage stream can be increased in an evaporation step where water is evaporated from the thin stillage. Concentrated thin stillage is referred to as syrup in the art. The syrup stream contains an increased concentration of corn oil, which can be separated and sold as distiller's corn oil (DCO). Alternatively, corn oil can be separated prior to fermentation, from the beer, from whole stillage, from thin stillage, from wet cake or any other corn oil containing process stream.

Biorefineries may separate DCO from process streams using centrifuges to produce a corn oil product. For example, U.S. Pat. No. 9,061,987 (Bootsma), U.S. Pat. No. 8,702,819 (Bootsma), and U.S. Pat. No. 9,695,449 (Bootsma), describe the separation of DCO using centrifuges, wherein the entireties of said patents are incorporated herein by reference. U.S. Pat. No. 8,008,516 (Cantrell et al.) describes DCO separation from thin stillage, wherein the entirety of said patent is incorporated herein by reference. U.S. Pat. No. 9,896,643 (Redford) reports methods and systems for recovering a desired co-product from a feedstock to ethanol production process, wherein the entirety of said patent is incorporated herein by reference.

While DCO is a valuable co-product, it is typically sold at commodity prices and used as a feedstock for biodiesel production or as an animal feed ingredient. There is a continuing need for refining grain oils such as corn oil and using grain oils for a variety of purposes. For example, there is a continuing need to refine grain oils by changing the color of one or more pigments that are present in the grain oil.

### SUMMARY

The present disclosure includes embodiments of a method of reducing the free fatty acid content in a grain oil. The method includes:

a) providing a first composition comprising a first grain oil portion, wherein the first composition has a first free fatty acid content based on the total weight of the first grain oil portion of the first composition;

b) exposing the first grain oil portion to an alcohol component and an esterase enzyme component to esterify at least a portion of the first free fatty acid content and form a second composition comprising a second grain oil portion, wherein the second composition has a second free fatty acid content based on the total weight of the second grain oil portion of the second composition, wherein the second free fatty acid content is less than the first free fatty acid content, wherein the esterase enzyme component is present in an amount from greater than 0 to 0.05% w/w of the weight of the first grain oil portion; and

c) separating the second composition or a composition derived from the second composition into an oil fraction and an aqueous fraction, wherein the second composition or the composition derived from the second composition has a first minerals content from 30 to 5,000 ppm, and the oil fraction has a second minerals content from 20 to 200 ppm, wherein the second minerals content is less than the first minerals content. In some embodiments, the gain oil having a reduced free fatty acid content can be used to form a grain oil product such as a bleached grain oil product.

The present disclosure also includes embodiments of a method of bleaching a grain oil product to provide a bleached grain oil product. The method includes:

a) providing a grain oil product including one or more pigments, wherein the grain oil product has an oxidative stability value of 7 hours or less according to AOCS Official Method Cd 12b-92-Reapproved 1997; and

b) contacting the grain oil product with an oxidizing gas under conditions to form a bleached grain oil product.

### BRIEF DESCRIPTION OF THE DRAWINGS

FIG. 1 is a schematic block diagram illustrating an embodiment of refining a grain oil composition feedstock according to the present disclosure;

FIG. 2 is a schematic block diagram of an embodiment of processing grain feedstock to form thin stillage and wet cake;

FIG. 3 is a schematic diagram of an embodiment of forming a grain oil composition feedstock from thin stillage;

FIG. 4 is a schematic process flow diagram illustrating an embodiment of refining a grain oil composition feedstock according to the present disclosure;

FIG. 5 is a schematic process flow diagram illustrating an embodiment of at least partially dehydrating an emulsion phase according to the present disclosure;

FIGS. 6A through 6G illustrate data from Example 5 and show volume percent foam reduction for seven different corn oil based antifoams at varying dose rates when using a model substrate (0.1 mass % sodium lauryl ether sulfate (SLES) in water);

FIG. 7A illustrates data from Example 6 and shows the volumetric percent foam reduction for a grain oil composition containing 90% ethyl esters, a grain oil composition containing 10% ethyl esters, six commercially available antifoam products, and food-grade refined Mazola corn oil when using a model substrate (0.1 mass % SLES in water);

FIG. 7B illustrates data from Example 6 and shows the volumetric percent foam reduction for a grain oil composition containing 90% ethyl esters, a grain oil composition containing 60% ethyl esters, a grain oil composition containing 10% ethyl esters, six commercially available antifoam products, and food-grade refined Mazola corn oil when using evaporated thin stillage from a cellulosic ethanol facility as substrate;

FIGS. 8A through 8G illustrate data from Example 8 and show that corn oil based antifoam compositions were effective at reducing foam in a sample of evaporated thin stillage from a cellulosic ethanol facility; and

FIG. 9 illustrates data from Example 9 as described below;

FIG. 10 illustrates an embodiment of bleaching a grain oil according to a batch process;

FIG. 11 illustrates an embodiment of bleaching a grain oil according to a continuous process;

FIG. 12 illustrates an embodiment of bleaching a grain oil according to a multi-stage continuous process;

FIG. 13 illustrates color change data from Example 14 below; and

FIG. 14 illustrates change in pigment concentration data from Example 14 below.

### DETAILED DESCRIPTION

The present disclosure involves grain oil compositions and byproducts thereof. As used herein, a “grain oil composition” refers to one or more compositions that have a grain oil portion and that can be used as a feedstock for the water refining process according to the present disclosure and one or more product compositions that have been refined according to the present disclosure. For example, a grain oil composition includes a grain oil composition feedstock such as distller's corn oil that is refined according to the present disclosure and a grain oil product produced thereby. In some embodiments, a grain oil composition can include a triglyceride component in an amount of at least 70 percent by weight of the total grain oil composition, at least 80 percent by weight of the total grain oil composition, at least 90 percent by weight of the total grain oil composition, or even at least 95 percent by weight of the total grain oil composition. A grain oil composition can also include a diglyceride component and/or monoglyceride component. In some embodiments, a grain oil composition can be derived from a fermentation product that has been produced via fermentation of a grain material. In some embodiments, a grain oil composition can include oil derived from oleaginous microorganisms.

As used herein, a “byproduct of a grain oil composition” refers to fractions or phases that are separated from a grain oil composition feedstock to form a grain oil product. As described herein below, nonlimiting examples of a byproduct of a grain oil composition include an emulsion phase and/or a dehydrated emulsion phase product and/or an aqueous phase. While one or more of these byproducts may include high levels of one or more triglycerides, diglycerides, and monoglycerides, they are byproducts of the refining process described herein.

In some embodiments a byproduct of a grain oil composition can have a triglyceride component present in an amount from 0 to 70 percent by weight of the total byproduct of a grain oil composition, or even from 5 to 50 percent by weight of the total byproduct of a grain oil composition.

As mentioned, the present disclosure includes embodiments of methods and systems for refining a grain oil composition feedstock to form a grain oil product. A method of refining a grain oil composition feedstock to provide a grain oil product includes providing a source of a grain oil composition feedstock.

A variety of grains (some of which may also be referred to as vegetables) can be used to provide a grain oil composition (and by-products thereof) such as one or more of corn, sorghum, wheat, rice, barley, soybean, rapeseed, oats, millet, rye and the like.

The grain oil composition feedstock includes at least a triglyceride component having one or more triglycerides. In some embodiments, the triglyceride component can be present in an amount of at least 70 percent by weight of the total grain oil composition feedstock, at least 80 percent by weight of the total grain oil composition feedstock, at least 90 percent by weight of the total grain oil composition feedstock, or even at least 95 percent by weight of the total grain oil composition feedstock. In some embodiments, the triglyceride component can be present in an amount from 70 to 99 percent by weight of the total grain oil composition feedstock, from 75 to 98 percent by weight of the total grain oil composition feedstock, from 80 to 95 percent by weight of the total grain oil composition feedstock, or even from 85 to 95 percent by weight of the total grain oil composition feedstock. Triglycerides can be determined by test method AOCS Cd 11d-96.

In some embodiments, the grain oil composition feedstock includes a diglyceride component having one or more diglycerides. In some embodiments, the diglyceride component can be present in an amount of 30 percent or less by weight of the total grain oil composition feedstock, 20 percent or less by weight of the total grain oil composition feedstock, 10 percent or less by weight of the total grain oil composition feedstock, or even 5 percent or less by weight of the total grain oil composition feedstock. In some embodiments, the diglyceride component can be present in an amount from 1 to 20 percent by weight of the total grain oil composition feedstock, from 1 to 15 percent by weight of the total grain oil composition feedstock, from 1 to 10 percent by weight of the total grain oil composition feedstock, or even from 1 to 5 percent by weight of the total grain oil composition feedstock. Diglycerides can be determined by test method AOCS Cd 11d-96.

In some embodiments, the grain oil composition feedstock includes a monoglyceride component having one or more monoglycerides. In some embodiments, the monoglyceride component can be present in an amount of 20 percent or less by weight of the total grain oil composition feedstock, 15 percent or less by weight of the total grain oil composition feedstock, 10 percent or less by weight of the total grain oil composition feedstock, or even 5 percent or less by weight of the total grain oil composition feedstock. In some embodiments, the monoglyceride component can be present in an amount from 1 to 15 percent by weight of the total grain oil composition feedstock, from 1 to 10 percent by weight of the total grain oil composition feedstock, from 1 to 5 percent by weight of the total grain oil composition feedstock, or even from 0.1 to 5 percent by weight of the total grain oil composition feedstock. Monoglycerides can be determined by test method AOCS Cd 11d-96.

In some embodiments, the grain oil composition feedstock includes a moisture content of 30 percent or less by weight of the total grain oil composition feedstock, 20 percent or less by weight of the total grain oil composition feedstock, 10 percent or less by weight of the total grain oil composition feedstock, 5 percent or less by weight of the total grain oil composition feedstock, or even 1 percent or less by weight of the total grain oil composition feedstock. In some embodiments, the moisture content can be from 0.01 to 10 percent by weight of the total grain oil composition feedstock, from 0.01 to 5 percent by weight of the total grain oil composition feedstock, from 0.01 to 1 percent by weight of the total grain oil composition feedstock, or even from 0.1 to 1 percent by weight of the total grain oil composition feedstock. Moisture content can be determined by a Karl Fischer titration (e.g., following ASTM E1064-12 or AOCS 2e-84).

The grain oil composition feedstock also includes an impurity component. As discussed below, the present disclosure includes methods and systems for removing at least a portion of the impurity component from the grain oil composition feedstock to produce a grain oil product having relatively higher purity, which can be more valuable. Depending on the intended use for the oil composition, one or more impurities can have an impact on one or more of oil color, catalyst fouling/inhibition (e.g., while forming biodiesel or renewable diesel from a grain oil composition feedstock), taste, smell, appearance, storage, and compatibility with further processing, materials and conditions to an undue degree. Accordingly, it may be desirable to remove one or more of these components. Nonlimiting examples of impurities include phospholipids, metals, free fatty acids, esters, soaps, gums, waxes, phosphatides, sterols, odiferous volatiles, colorants, and combinations thereof.

In some embodiments, grain oil composition feedstock includes an impurity component that includes at least an element component having one or more elements chosen from aluminum, arsenic, cadmium, calcium, chlorides, chromium, copper, iron, lead, magnesium, manganese, mercury, nitrogen, nickel, phosphorus, potassium, silicon, sodium, sulfur, vanadium, zinc, and combinations thereof. In some embodiments, the impurity component includes at least one element chosen from calcium, phosphorus, potassium, sodium, and combinations thereof. Metals can be determined by test method AOCS Ca 17-01. Phosphorus can be determined by test method AOCS Ca 20-99. Sulfur can be determined by test method ASTM D4951.

In some embodiments, the grain oil composition feedstock includes the element component in an amount of 100 parts per million (ppm) or more based on the total grain oil composition feedstock, 200 ppm or more based on the total grain oil composition feedstock, 500 ppm or more based on the total grain oil composition feedstock, 1000 ppm or more based on the total grain oil composition feedstock, or even 5000 ppm or more based on the total grain oil composition feedstock. In some embodiments, the element component can be from 5 to 10,000 ppm based on the total grain oil composition feedstock, from 100 to 5000 ppm based on the total grain oil composition feedstock, or even from 500 to 1000 ppm based on the total grain oil composition feedstock.

In some embodiments, at least a portion (e.g., including substantially all) of the element component is present as soap, which is a salt of the element and a fatty acid such as sodium oleate, magnesium stearate, combinations of these, and the like. In some embodiments, grain oil composition feedstock includes a soap component in an amount from 50 to 30,000 ppm, from 100 to 20,000 ppm, or even from 500 to 10,000 ppm. Soap content can be determined by test method AOCS Cc17-95.

In some embodiments, the grain oil composition feedstock contains no detectable phospholipid. For example, any phospholipid that may have been inherently present in the raw grain material may have been removed in an upstream process.

A grain oil composition feedstock can also include a fatty acid alkyl ester (FAAE) component including one or more fatty acid alkyl esters such as fatty acid ethyl ester (FAEE), which is an esterified (not free) fatty acid. For example, FAEE is a fatty acid esterified with ethanol as a free fatty acid becomes exposed to ethanol during fermentation and through distillation in a biorefinery. A fatty acid alkyl ester component includes one or more fatty acid alkyl esters. Nonlimiting examples of fatty acid ethyl esters include one or more of ethyl linoleate, ethyl linolenate, ethyl oleate, ethyl palmitate, and ethyl stearate.

A grain oil composition feedstock can also include a free fatty acid component including one or more free fatty acids. As used herein a “free fatty acid” refers to an unesterified fatty acid, or more specifically, a fatty acid having a carboxylic acid head and a saturated or unsaturated unbranched aliphatic tail (group) of from 4 to 28 carbons. The term “aliphatic” has a generally recognized meaning and refers to a group containing only carbon and hydrogen atoms which is straight chain, branched chain, cyclic, saturated or unsaturated but not aromatic. A free fatty acid component includes one or more free fatty acids. Nonlimiting examples of free fatty acids include, e.g., caproic acid, capric acid, caprylic acid, lauric acid, myristic acid, palmitic acid, stearic acid, oleic acid, linolenic acid, linoleic acid, arachidic acid, and mixtures thereof.

In some embodiments, a grain oil composition feedstock is derived from a fermentation product, or beer. Fermentation products can be produced by hydrolyzing materials containing sugar polymers and oil to produce a fermentable material containing fermentable sugars and oil and converting the sugars into a fermentation product using an organism capable of fermentation.

For example, a grain containing starch and oil may be ground and the starch hydrolyzed into fermentable sugars, e.g., by using one or more enzymes, chemicals, heat, and/or other catalyst. The fermentable sugars may be converted into a target chemical, e.g. an alcohol such as ethanol using an organism, e.g. an ethanologen. The fermentation product can include the target chemical e.g. ethanol, water, oil, additional soluble components, unfermented particulate matter, and the like. The fermentation product can then be distilled to recover the target chemical, e.g. ethanol, leaving the remaining components as whole stillage.

A fermentation product containing oil can be derived from one or more grain materials. A variety of grain materials (some of which may also be referred to as vegetable materials) can be used such as whole ground grain or a fraction of a grain. Grain material can be derived from grain such as corn, sorghum, wheat, rice, barley, soybean, rapeseed, oats, millet, rye or any other grains that that are capable of being fermented and subjected to the refined oil process described herein.

In some embodiments, oil in the fermentation product can also be derived from oleaginous microorganisms. Exemplary oleaginous microorganisms include oleaginous microalgae, which can include the genus Chlorella or Prototheca, including, Chlorella protothecoides or Prototheca moriformis, Nannochloropsis salina, Chlorella vulgaris, Scenedesmus dimorphus, and Chaetoceros gracilis. Other exemplary oleaginous microorganisms include yeast such as Yarrowia lipolytica, Cryptococcus curvatus, Rhodosporidium toruloides, and bacteria such as Rhodococcus opacus.

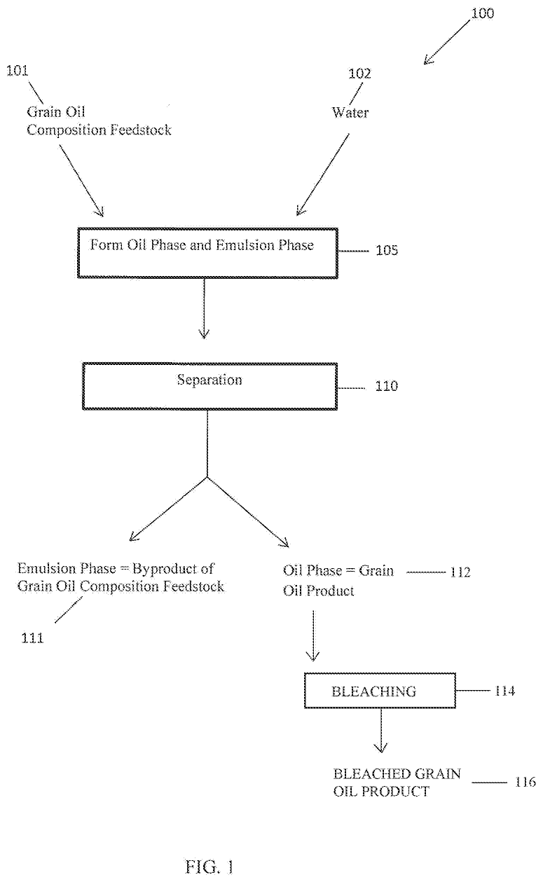

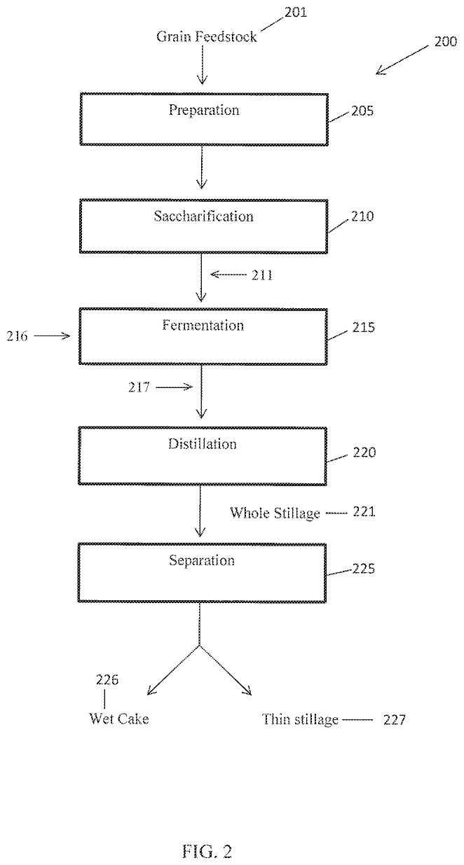

For illustration purposes, a process for obtaining a corn oil composition feedstock from corn grain is described herein. The process includes preparing the corn, saccharifying sugar polymers to obtain fermentable sugars, fermenting the sugars, recovering a corn oil composition feedstock, and refining the corn oil composition feedstock to form a corn oil product. A nonlimiting example of providing a corn grain oil composition feedstock for refining according to the present disclosure is illustrated in FIGS. 1 and 2.

Preparation of Grain for Saccharification

As shown in FIG. 2, process 200 includes providing grain feedstock 201, e.g. corn, that is first be prepared 205 for saccharification 210 by reducing the size of the grain. In some embodiments, corn grain can be dry milled (e.g., hammer milled) to produce whole ground corn having a medium-to-fine grind for use in saccharification. In some embodiments, corn grain can be dry-fractionated to separate components of the corn grain (e.g., germ) from each other and then recombine two or more components (e.g., the endosperm and germ) for saccharification.

In some embodiments, the corn grain can be ground so that a substantial portion, e.g., a majority, of the ground corn grain fits through a sieve with a 0.1-5.0 mm screen, or even a 0.1-9 0.5 mm screen. In some embodiments, 50% or more, 60% or more, or even 70% or more of the ground corn can fit through a sieve with a 0.1-0.5 mm screen.

Ground corn can be mixed with an appropriate amount of water to form an aqueous composition (e.g., a slurry) for subsequent saccharification of the slurry and fermentation of the resulting sugars. In an embodiment, whole ground corn can be mixed with liquid at about 20 to about 50 wt-% or about 25 to about 45 wt-% dry whole ground corn. The whole ground corn can include starch, fiber, protein, oil, endogenous enzymes, amino acids, etc. Any corn grain components (e.g., residual fiber, starch, sugar, oil, etc.) remaining after fermentation can be extracted/separated after fermentation and/or distillation, as discussed below. Because starch constitutes the largest mass portion of the corn grain it can be more efficient to extract other components (e.g., oil, fiber, protein, etc.) after at least a portion of the starch has been removed (i.e., hydrolyzed into glucose which is consumed by, e.g., yeast).

Saccharification

After forming an aqueous slurry that includes the corn material from preparing corn as described above, the aqueous slurry can be subjected to saccharification 210 to break down (hydrolyze) at least a portion of the starch into glucose that can be used by yeast during fermentation.

Saccharification can be performed by a variety of techniques. For example, heat and/or one or more enzymes can be used to saccharify components of the prepared corn into oligomers and monomers.

In some embodiments, starch can be saccharified using a conventional, relatively high temperature cooking process.

In some embodiments, a relatively low temperature saccharification process involves enzymatically hydrolyzing at least a portion of the starch in the aqueous slurry at a temperature below starch gelatinization temperatures, so that saccharification occurs directly from the raw native insoluble starch to soluble glucose while bypassing conventional starch gelatinization conditions. Starch gelatinization temperatures are typically in a range of 57° C. to 93° C. depending on the starch source and polymer type. Converting raw starch to glucose with one or more exogenous enzymes, e.g., glucoamylase and acid fungal amylase is described in U.S. Pat. Nos. 30 7,842,484 (Lewis) and 7,919,291 (Lewis et al.), wherein the entireties of the patents are incorporated herein by reference. In one embodiment, saccharification includes enzymatically (e.g., with alpha-amylases and gluco-amylases) hydrolyzing at least a portion of the starch in the aqueous slurry at a temperature of 40° C. or less and a pH (e.g., from 3 to 6) to produce a slurry that includes glucose. In some embodiments, enzymatic hydrolysis occurs at a temperature in the range of from 25° C. to 35° C. to produce a slurry that includes glucose.

In some embodiments, a composition including 20% to 50 wt % ground corn having a corn oil portion, and at least one glucoamylase and/or at least one fungal acid amylase, is exposed to conditions which produce glucose. In some embodiments, the amount of the fungal acid amylase ranges from 0.1 to 10 fungal acid amylase units per gram of solids in the composition, and the amount of the glucoamylase to solids in the composition ranges from 0.5 to 6 glucoamylase units per gram of said solids.

In some embodiments, saccharification of starch can include heating the slurry to a temperature in the range from 50° C. to 100° C.; from 60° C. to 90° C.; or even from 80° C. and 85° C. and adding a thermostable alpha-amylase to the slurry to initiate liquefaction. In some embodiments, saccharification of the starch can include jet-cooking the slurry at a temperature between 100° C. to 145° C. to complete gelatinization of the slurry.

Fermentation

After saccharification, the resulting slurry (“grain mash composition”) includes grain solids, grain oil and sugar. The sugar (glucose) that is generated from saccharification can be fermented 215 into a “beer” composition that includes one or more biochemicals (e.g., butanol, ethanol, and the like). Systems for producing more than one biochemical from the glucose can be integrated together or be separate. Fermenting can be carried out by microorganisms. Exemplary microorganisms include ethanologens, butanologens, and the like. Exemplary microorganisms include yeasts.

In some embodiments, fermenting can include contacting an aqueous slurry including sugars derived from ground corn with microorganisms under conditions suitable for growth of the microorganisms and production of a biochemical. For examples, yeasts may be used that convert the sugars to ethanol. Suitable yeasts include any variety of commercially available yeasts, such as commercial strains of Saccharomyces cerevisiae.

Optionally, one or more components (e.g., yeast nutrients) can be included in the aqueous slurry that is to be fermented.

In some embodiments, saccharification and fermentation can occur simultaneously in the same reactor (also referred to as simultaneous saccharification and fermentation (SSF)).

In some embodiments, fermentation includes fermenting an aqueous slurry in the presence of yeast under conditions which produce a composition (“beer”) that includes ethanol and a corn oil portion having a fatty acid ethyl ester content as described herein.

Esterase Enzyme

In some embodiments, an esterase enzyme component can be present in a composition that includes grain oil to at least catalyze the esterification of one or more free fatty acids in the presence of alcohol (e.g., one or more alcohols such as ethanol) into one or more fatty acid alkyl esters (e.g., one or more fatty acid ethyl esters) and reduce the concentration of the free fatty acids in the grain oil as compared to if the grain oil was not treated with the esterase component (e.g., not treated with exogenous esterase enzyme component).

An esterase enzyme component can include one or more esterase enzymes (e.g.., an enzyme “cocktail”). An esterase enzyme component can include one or more esterase enzymes chosen from one or more endogenous enzymes, one or more exogenous enzymes, and combinations thereof. Non-limiting examples of esterase enzymes includes lipase enzymes, hydrolase enzymes, and combinations thereof. In some embodiments, an esterase enzyme component can include exogenous triacylglycerol lipase, exogenous carboxylic ester hydrolase, and combinations thereof. Esterase enzymes are commercially available, for example, from Novozymes under the tradename Eversa®Transform 2.0 enzyme, and under the tradename Lipozyme® CALB L lipase enzyme.

Table 1 below illustrates how fermenting in the presence of an esterase enzyme such as lipase can change the content of free fatty acids by esterifying the free fatty acids in the presence of ethanol to form fatty acid ethyl esters. It is noted that the first four columns from the left in Table 1 report the dosing of the lipase enzyme in different units.

TABLE 1
Lipozyme CALB L lipase enzyme approximate dosing
and corresponding FAEE and FFA data in the oil.

| | | % w/w | | | |
|---|---|---|---|---|---|
| LU/g | % w/w | Corn | L/550,000 | % FAEE | FFA |
| DS^{a} | DS | Oil | gal Ferm | (% w/w)^{b} | (% w/w)^{b} |
| 0 | 0% | 0% | 0 | 13.0% | 17.9% |
| 0.033 | 0.0006% | 0.02% | 5 | 18.6% | 14.1% |
| 0.067 | 0.0013% | 0.04% | 10 | 21.3% | 10.6% |
| 0.323 | 0.0065% | 0.202% | 50 | 29.3% | 4.3% |
| 0.65 | 0.0130% | 0.404% | 100 | 31.2% | 3.9% |

^{a}Lipase Unit (LU), Dry Solids (DS)
^{b}FFA and FAEE content of oil solvent extracted from the emulsion

It has been observed that the content of free fatty acid in oil can be correlated to the content of metal soaps that are present in a grain oil composition (e.g., a grain oil product). For example, when breaking an oil-water emulsion by increasing the pH (e.g., with sodium hydroxide) one or more metal soaps can be formed. During subsequent separation of, e.g., a grain oil product from defatted emulsion such as defatted emulsion 316 discussed below, at least some of these soaps can be carried over into the grain oil product and thereby increase the metal content of the grain oil product even though free metals tend to go with the defatted emulsion 316. For example, the metal content of soaps can contribute to a relatively high density for soap such that the soap tends to go with the defatted emulsion, which is mostly water on a volume basis, even if the soap is water soluble or water insoluble (e.g., sodium-based and potassium-based soaps tend to be water soluble while calcium-based and magnesium-based soaps tend to be water insoluble). But, if the soap content is too high, some soap can “spill over” into the grain oil product when it is separated from the defatted emulsion. As the content of free fatty acids in an oil emulsion is reduced, the content of metal soap has been observed to also be relatively lower due to less metal soap being formed. With less soap formation, there is less soap to be carried and consequently less resulting metal content in the grain oil product. However, it has also been observed that treating an oil composition with too much esterase enzyme component can result in an increase of metal soaps in the oil as compared to if no esterase enzyme treatment had been performed on the oil. While not being bound by theory, it is believed that this may result because as the FAAE content of the oil increases its viscosity decreases leading to a smaller difference between the viscosities of the oil and the defatted emulsion (e.g., defatted emulsion 316) thereby making a clean separation more difficult and resulting in some of the aqueous phase, including soaps and possibly free metals being carried into the oil phase. In some embodiments, a given separation technique (e.g., centrifuge) may be able to be adjusted to accommodate a shift in viscosities among the oil and defatted emulsion to facilitate making a more clean break when using high doses of lipase. It may also be the result of increasing difficulty in breaking the emulsion with higher lipase doses because the emulsion is relatively more stable. As yet another theory, it is believed that this may result because at higher lipase dose, the lipase favors free fatty acid formation relative to esterification of free fatty acids in the presence of alcohol.

For example, as shown in Table 2 below the amount of free fatty acid increased when treated with 50 L/550,000 gal of exogenous lipase as compared to if the oil had not been treated with an exogenous esterase enzyme component for a given set of conditions. It is noted that the data in Table 2 represents oil recovered at an ethanol plant after treatment with the lipase enzyme during fermentation in the presence of ethanol and after treatment of a recovered oil-water emulsion with sodium hydroxide, which formed metal soaps.

TABLE 2
Effect of Exogenous Lipase Enzyme Dosing
on FFA and Total Phosphorus and Metals

| | | | | | Total |
|---|---|---|---|---|---|
| Lipase Dose | | Approximate | Approximate | % FFA | Phosphorous |
| (L/550.000 gal) | Lipase Name | pH | Hours | and in Oil | Metals |
| 0 | NA | 4.5 | 70 | 4.2% | 151 |
| | Novozymes | | | | |
| | Eversa | | | | |
| 50 | Transform 2.0 | 4.6 | 70 | 8.9% | 3299 |
| 0 | NA | 4.3 | 80 | 7.3% | 276 |
| | Novozymes | | | | |
| | Lipozyme | | | | |
| 5 | CALB L | 4.3 | 80 | 4.2% | 57 |
| 0 | NA | 4.6 | 90 | 4.8% | 224 |
| | Novozymes | | | | |
| | Lipozyme | | | | |
| 3 | CALB L | 4.6 | 90 | 3.8% | 51 |

By treating free fatty acids with an esterase enzyme component according to the present disclosure to esterify at least a portion of the free fatty acids and reduce the free fatty acid content, the metal soap content, and therefore minerals content (metals and phosphorus), in a grain oil product (e.g., a bleached grain oil product) can advantageously be managed.

The free fatty acid content in a composition prior to treating with an esterase enzyme component according to the present disclosure is higher as compared to the free fatty acid content in the composition after treating with an esterase enzyme component according to the present disclosure. In some embodiments, the free fatty acid content in a composition prior to treating with an esterase enzyme component according to the present disclosure is from 2 to 30% w/w based on the total weight of the grain oil portion of the composition, from 2 to 25% w/w based on the total weight of the grain oil portion of the composition, or even from 2 to 20% w/w based on the total weight of the grain oil portion of the composition. Free fatty acid can be determined by test method AOCS Ca 5a-40.

The fatty acid alkyl ester content in a composition prior to treating with an esterase enzyme component according to the present disclosure is lower as compared to the fatty acid alkyl ester content in the composition after treating with an esterase enzyme component according to the present disclosure because an esterase enzyme component treatment according to the present disclosure esterifies free fatty acid to form fatty acid alkyl ester. In some embodiments, the fatty acid alkyl ester content prior to treating with an esterase enzyme component according to the present disclosure is 20% or less w/w based on the total weight of the grain oil portion of the composition. In some embodiments, the free fatty acid content in a composition after treating with an esterase enzyme component according to the present disclosure is from greater than 0 to 10% w/w based on the total weight of the grain oil portion of the composition, from greater than 0 to 5% w/w based on the total weight of the grain oil portion of the composition, from greater than 0 to 4% w/w based on the total weight of the grain oil portion of the composition, from greater than 0 to 3% w/w based on the total weight of the grain oil portion of the composition, or even from greater than 0 to 1% w/w based on the total weight of the grain oil portion of the composition. In some embodiments, the fatty acid alkyl ester content in a composition prior to treating with an esterase enzyme component according to the present disclosure is from 0.01 to 10% w/w based on the total weight of the grain oil portion of the composition. Fatty acid alkyl ester content can be determined by the following test method, which is illustrated in the context of ethyl esters:

Fatty Acid Ethyl Ester Determination by Gas Chromatography

Approximately 50 mg of the extracted oil can be added to a 10 mL volumetric flask to which xylene can be added to the 10 ml_, graduation. External standards including ethyl palmitoleate, ethyl oleate, and ethyl linoleate can be used to generate standard curves to determine the amount of each individual fatty acid ethyl ester present in the extracted corn oil. Standard concentrations can range from 0.02 mg mL¹ to 0.40 mg mL¹. The samples and standards can be run on a gas chromatograph (GC) equipped with a split/splitless injector (with splitless glass liner) and flame ionization detector (HD). Also, the GC can be equipped with a Phenomenex Zebron ZB-Waxplus column (30mL×0.32 mm ID×0.25 μτη df). Analysis can be conducted by injecting 1 μL of the sample into the inlet held at 250° C. The oven can be initially set at 170° C. and followed an oven temperature gradient of 2° C. min⁻¹ up to 200° C. holding for 15 minutes, followed by a temperature gradient of 5° C. min⁻¹ up to 230° C. holding for nine minutes.

The detector was maintained at a temperature of 250° C. Hydrogen was used as the carrier gas and the flow was controlled in constant flow mode at 1.80 mL

Major ethyl esters in corn oil are ethyl palmitate, ethyl stearate, ethyl oleate, ethyl linoleate, and ethyl linolenate of FAEE. With the aforementioned instrument parameters, ethyl palmitate would elute around 11 minutes, ethyl stearate around 16.5 minutes, ethyl oleate around 7 minutes, ethyl linoleate around 18 minutes, and ethyl linolenate around 19.5 minutes. A standard curve of each ethyl ester can be obtained to give the slope and y-intercept for quantitation. Ethyl palmitate concentration can be determined by the ethyl palmitoleate standard curve, ethyl stearate and ethyl oleate concentration can be determined by the ethyl oleate standard curve, and ethyl linoleate and ethyl linolenate concentrations can be determined from the ethyl linoleate standard curve. The total FAEE content of each sample can be determined using the equation below.

$$\% FAEE ( \% mg \text{/} mg ) = \sum \frac{A \text{?} - y \text{?} \cdot 10}{S \cdot m}$$

$$\text{?} \text{indicates text missing or illegible when filed}$$

Where:

[symbol]

An esterase enzyme component can be present in a range of concentrations so that it reduces the fatty acid content of the composition (e.g., as described above with respect to Table 2). The amount of esterase enzyme component to produce a desired reduction in free fatty acid content (and reduction in subsequent metal soap formation) can vary depending on factors such as one or more of the enzyme component that is selected, the pH during treatment, and the time period of treatment, the amount of endogenous esterase enzyme present and combinations thereof. It is noted that a conventional high temperature cooking process to saccharify starch can reduce the amount of active endogenous esterase enzyme as compared to the raw starch process described above and, therefore, a relatively higher dose of exogenous esterase enzyme component may be used for a composition that has undergone a conventional high temperature cooking process to saccharify starch as compared to composition that has undergone a raw starch process. As described below, the concentration of the of the esterase enzyme component is based upon the total weight of corn oil present in a composition (e.g., the total weight of corn mash in a fermenter or the total weight of a grain oil composition). In some embodiments, an esterase enzyme component is present in an amount from greater than 0 to 0.05% w/w of the weight of a grain oil portion of a composition; from greater than 0 to 0.04% w/w of the weight of a grain oil portion of a composition; from greater than 0 to 0.03% w/w of the weight of a grain oil portion of a composition; from greater than 0 to 0.02% w/w of the weight of a grain oil portion of a composition; from greater than 0 to 0.01% w/w of the weight of a grain oil portion of a composition; from greater than 0 to 0.005% w/w of the weight of a grain oil portion of a composition; from greater than 0 to 0.0025% w/w of the weight of a grain oil portion of a composition; or even from greater than 0 to 0.001% w/w of the weight of a grain oil portion of a composition. In some embodiments, an esterase enzyme component is present in an amount of at least 0.0001% w/w of the weight of a grain oil portion of a composition; at least 0.001% w/w of the weight of a grain oil portion of a composition; or even at least 0.002% w/w of the weight of a grain oil portion of a composition. These concentrations can result from one or more esterase enzymes that are naturally present in grain, generated endogenously via one or more microorganisms, exogenously added, or a combination thereof

A composition can be treated with an esterase enzyme component under conditions to reduce the fatty acid content in the composition, which can depend on the esterase enzyme component selected. In some embodiments, an esterase enzyme component can be exogenously added to one or more of a grain mash composition prior to fermentation, a grain mash composition during fermentation, a beer composition, whole stillage, thin stillage, condensed thin stillage, distiller's corn oil, any composition/process stream derived therefrom and combinations thereof, and exposed to conditions to at least esterify one or more free fatty acids, thereby reducing the total free fatty acid content. Referring to FIG. 2, in some embodiments, when exogenously added, the esterase enzyme component can be added to a composition having a grain oil portion before fermentation 211, during fermentation 216, after fermentation 217, or combinations thereof.

The conditions during treatment with esterase enzyme component may be selected depending on, for example, when in the overall process a composition is treated with esterase enzyme component. As mentioned above, treatment with esterase enzyme component can occur at one or more points in an overall ethanol biorefinery where grain oil product is recovered in addition to ethanol. In some embodiments, conditions during treatment with esterase enzyme component can be selected as fermentation conditions that are based on the microorganism selected for fermentation. If the conditions during treatment with esterase enzyme component are less than optimal for the given esterase enzyme component, the dosage of esterase enzyme component may be adjusted (e.g., increased) to accommodate such conditions as long as the benefit of treatment with esterase enzyme component is achieved, which is the reduction of free fatty acid content and metal soap content as compared to if no treatment with esterase enzyme component was used. In some embodiments, conditions during treatment with esterase enzyme component can be selected as optimal for esterification of free fatty acid by the esterase enzyme component.

An example of including an esterase enzyme to convert free fatty acid into fatty acid alkyl ester in an oil composition is described in U.S. Patent Publication 2018/0340197 (McCurdy et al.), wherein the entirety of said patent publication is incorporated herein by reference. An example of fermenting grain mash in the presence of one or more enzymes (endogenous enzymes and/or exogenous enzymes) such as lipase, esterase, and combinations thereof to generate one or more fatty acid alkyl esters is described in U.S. Patent Publication 2019/0376002 (Urban et al.), wherein the entirety of said patent publication is incorporated herein by reference.

As mentioned, the fatty acid alkyl ester content in a composition after treating with an esterase enzyme component according to the present disclosure (and prior to any further treatment) is higher as compared to the fatty acid alkyl ester content in the composition prior to treating with an esterase enzyme component according to the present disclosure. In some embodiments, the fatty acid alkyl ester content in a composition after treating with an esterase enzyme component according to the present disclosure is 5% w/w or greater based on the total weight of the grain oil portion of the composition from 5 to 40% w/w based on the total weight of the grain oil portion of the composition, from 10 to 40% w/w based on the total weight of the grain oil portion of the composition, from 10 to 25% w/w based on the total weight of the grain oil portion of the composition, or even from 15 to 25% w/w based on the total weight of the grain oil portion of the composition.

The free fatty acid content in a composition after treating with an esterase enzyme component according to the present disclosure (and prior to any further treatment) is lower as compared to the free fatty acid content in the composition prior to treating with an esterase enzyme component according to the present disclosure. In some embodiments, the free fatty acid content in a composition after treating with an esterase enzyme component according to the present disclosure is 10% or less w/w based on the total weight of the grain oil portion of the composition. In some embodiments, the free fatty acid content in a composition after treating with an esterase enzyme component according to the present disclosure is from greater than 0 to 10% w/w based on the total weight of the grain oil portion of the composition, from greater than 0 to 5% w/w based on the total weight of the grain oil portion of the composition, from greater than 0 to 3% w/w based on the total weight of the grain oil portion of the composition, from greater than 0 to 1% w/w based on the total weight of the grain oil portion of the composition, or even from greater than 0 to 0.5% w/w based on the total weight of the grain oil portion of the composition. In some embodiments, the free fatty acid content in a composition prior to treating with an esterase enzyme component according to the present disclosure is from 0.01 to 10% w/w based on the total weight of the grain oil portion of the composition. Free fatty acid can be 3determined by test method AOCS Ca 5a-40.

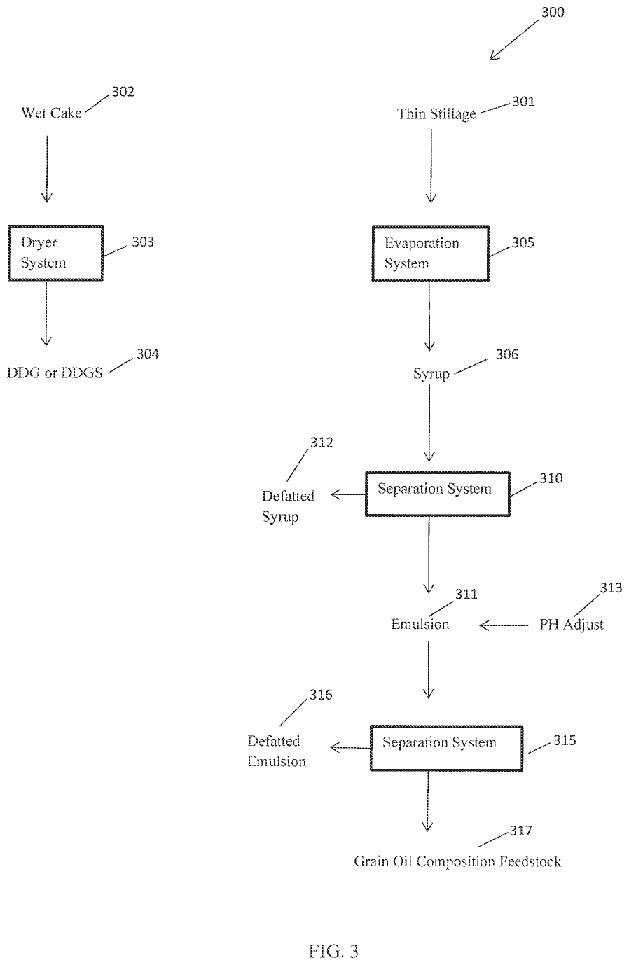

As mentioned above, because the free fatty acid content can be reduced after being treated with an esterase enzyme component according to the present disclosure, the potential to form metal soaps is thereby also reduced. This reduced potential to form metal soaps can be for any pH, but in some embodiments the tendency to form metal soaps can be higher at a relatively higher pH. For example, a composition can be present as an emulsion. After treating the composition with esterase enzyme component as described herein the composition can subsequently have its pH raised to help break the emulsion. An example of breaking an emulsion is discussed below with respect to FIG. 3 and emulsion 311. Raising the pH of the composition in such a manner can cause formation of metal soap between a metal ion and free fatty acid. In some embodiments, a composition can have its pH raised from 6 less, 5 or less, or even 4 or less. Its pH can be raised to 7 or greater, 7.5 or greater, 8 or greater, or even 8.5 or greater. Such a composition can have its pH adjusted by, e.g., contacting it with an alkali base such as calcium hydroxide, sodium hydroxide, potassium hydroxide, rubidium hydroxide, caesium hydroxide, and combinations thereof. But, because the free fatty acid content can be reduced after being treated with an esterase enzyme component according to the present disclosure, the potential to form metal soaps is thereby also reduced.

A composition after being treated with an esterase enzyme component according to the present disclosure and prior to having its pH adjusted as just described can have a minerals content present in an amount that is higher as compared to the minerals content of the grain oil composition that is recovered from the pH adjusted composition because the minerals content tends to go with the defatted emulsion.

As used herein, “minerals content” refers to the total metals content and total phosphorus content. In some embodiments, metals include one or more metals chosen from aluminum, arsenic, cadmium, calcium, chlorides, chromium, copper, iron, lead, magnesium, manganese, mercury, nitrogen, nickel, potassium, silicon, sodium, vanadium, zinc, and combinations thereof. Metals can be determined by test method AOCS Ca 17-01. Phosphorus can be determined by test method AOCS Ca 20-99.

In some embodiments, the minerals content of a composition after being treated with an esterase enzyme component according to the present disclosure and after having its pH adjusted as just described can be from 50 to 10,000 ppm, from 30 to 5,000 ppm, or even from 100 to 3,000 ppm.

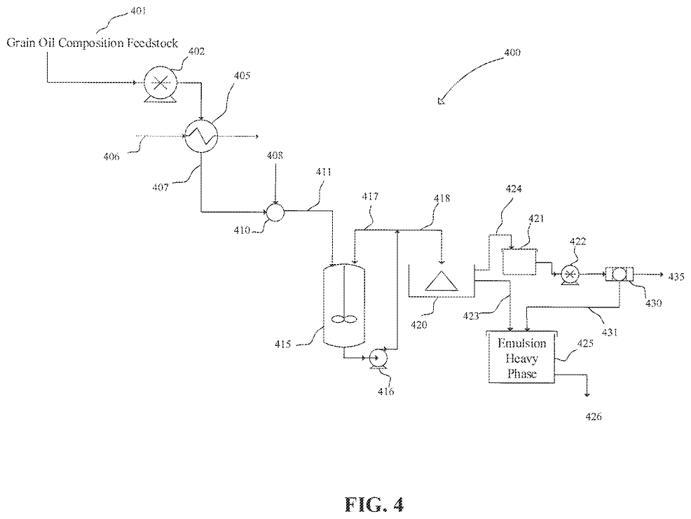

In some embodiments, the minerals content of a grain oil composition recovered from the composition that was treated with an esterase enzyme component according to the present disclosure and after having its pH adjusted as just described can be from 20 to 200 ppm, from 10 to 100 ppm, or even from 10 to 50 ppm. Such a grain oil composition can be a grain oil composition feedstock that can be refined using water as described in FIG. 4 below and have its impurity component (including minerals content) reduced even further, e.g., below 10 ppm.

Distillation and Post-Distillation

After fermentation, the biochemical can be removed from the beer in a distillation system 220 to form a whole stillage 221. For example, a beer derived from corn can be distilled to remove ethanol and form whole stillage. For example, heat and/or vacuum may be applied to the fermentation product in a distillation unit to evaporate and condense the biochemical to separate it from the rest of the fermentation product. The bottoms stream from the distillation unit after the biochemical has been recovered is referred to as whole stillage 221. This whole stillage stream 221 includes, e.g., suspended solids, dissolved solids, water, and oil. The whole stillage stream is separated, typically by decanting centrifuges, into a thin stillage stream 227 and a wet cake stream 226.

The wet cake stream 226 is a wet, solid stream e.g. greater than 25% solids w/w. The thin stillage stream 227 is a liquid stream that contains a lower concentration of suspended solids, e.g. less than 15% solids w/w, compared to whole stillage.

As mentioned above, in some embodiments, an esterase enzyme component can be exogenously added to treat a composition after distillation such as whole stillage, thin stillage, condensed thin stillage, distiller's corn oil, and combinations thereof, and exposed to conditions to at least esterify one or more free fatty acids, thereby reducing the total free fatty acid content of the composition. If alcohol such as ethanol has been removed via distillation then alcohol can be added to the composition (e.g., recycled ethanol that has been removed via distillation) to facilitate esterification of free fatty acid into fatty acid alkyl ester. In some embodiments, treating with esterase enzyme component after distillation can include conditions that are relatively more optimal to esterify the free fatty acid content in a manner that reduces the overall free fatty acid content. In some embodiments, conditions to enzymatically esterify at least a portion of the free fatty acid content via an esterase enzyme component in a manner that reduces the overall free fatty acid content include a pH from 4.5 to 6.5 (or even from 5 to 6) at a temperature from 40° C. to 60° C. (or even from 45° C. to 55° C.) for a time period from 6 hours to 96 hours (or even from 24 hours to 60 hours).

Referring now to the nonlimiting, exemplary process flow 300 in FIG. 3, the thin stillage stream 301 and wet cake stream 302 can be further processed as illustrated. The solids concentration of the thin stillage stream can be increased in an evaporation step 305 where water is evaporated from the thin stillage 301. Concentrated thin stillage is referred to as syrup 306 in the art. The syrup stream 306 contains an increased concentration of corn oil, which can be separated as an oil composition and sold as distiller's corn oil (DCO).

In some embodiments, an oil composition can be derived from a fermentation product before distillation. In some embodiments, an oil composition can be derived from a fermentation product after distillation. For example, in some embodiments an oil composition can be derived from a whole stillage composition obtained after distillation of a fermentation product.

The wet cake 302 can be dried in a dryer system 303 to provide “Distillers Dried Grains” (DDG) 304. In some embodiments, a portion of the syrup may be blended into DDG or added to the wet cake before drying to produce Distillers Dried Grain with Solubles (DDGS).

As used herein, “grain stillage composition” is used to refer to whole stillage, thin stillage, wet cake and/or syrup. A grain oil composition feedstock may be derived from the grain stillage composition.

Referring to FIG. 3, syrup 306 can be processed to provide a grain oil composition feedstock 317 that can be refined with water according to the present disclosure. As shown in FIG. 3, syrup can be separated via separation system 310 into a first oil fraction 311 and a first aqueous fraction 312 (defatted syrup). The first oil fraction 311 can be referred to as distiller's corn oil. In some embodiments, first oil fraction 311 is a grain oil composition feedstock that is refined with water according to the present disclosure. As shown, if first oil fraction 311 is an emulsion, it can have its pH adjusted to break the emulsion into a second oil fraction 317 and a second aqueous fraction 316 (defatted emulsion). In some embodiments, emulsion 311 is at a pH in the range from 3 to 4 and its pH is increased to a pH from 7 to 9, for example, by adding a basic material 313 such as sodium hydroxide. In some embodiments, as shown, the second oil fraction 317 is a grain oil composition feedstock refined with water according to the present disclosure. The second oil fraction can also be referred to as distiller's corn oil that is sold under the trade name Voila® corn oil.

Examples of methods of extracting oil from a stillage composition are described at U.S. Pat. No. 9,061,987, (Bootsma), U.S. Pat. No. 8,702,819 (Bootsma), and U.S. Pat. No. 9,695,449 (Bootsma) wherein oil is separated using centrifuges. The entireties of these patents are incorporated herein by reference. U.S. Pat. No. 8,008,516 (Cantrell et al.) describes DCO separation from thin stillage, wherein the entirety of the patent is incorporated herein by reference. U.S. Pat. No. 9,896,643 (Redford) describes recovering a light phase product from ethanol product, wherein the entirety of the patent is incorporated herein by reference.

Optionally, a grain oil composition feedstock can be treated before being refined according to the present disclosure. Nonlimiting examples of such treatments include one or more of acid degumming, adding a flocculating agent to the grain oil composition, adding a filter aid to the grain oil composition. In some embodiments, the grain oil composition feedstock may be acid degummed according to commercially available processes to remove gums present in the grain oil composition feedstock. As part of a degumming process, an acid, such as phosphoric acid, may be added to the grain oil composition feedstock and the grain oil composition feedstock may be heated, for example, using steam. In such a process, the acid and steam work to swell the gums so that the gums can be separated from the grain oil composition feedstock, such as by centrifugation or another suitable separation technique. In some embodiments, the addition of the strong base can be used to neutralize the free fatty acids may occur after addition of the acid in the acid degumming step. In this embodiment, the base added to neutralize the free fatty acids can also work to neutralize the acid used in the acid degumming step. The soap stock that results from acid degumming and neutralization of the grain oil composition feedstock may be separated from the grain oil composition feedstock using standard equipment, such as a centrifugal separator. Alternatively, the free fatty acids can be removed and acid esterified to form bio-diesel, or combined with glycerin to form triglycerides, which are then transesterified to form bio-diesel. Prior to any transesterification process, the acid degummed and neutralized oil can be washed. Washing may include, for example, mixing with warm wash water. After washing, the grain oil product and wash water are separated, and the grain oil product is dried, such as by a vacuum-dryer, to a desired water content. Acid degumming is further described in in U.S. Patent Publication 2020/0063168 (Bootsma), wherein the entirety of said patent publication is incorporated herein by reference.

In some embodiments, a grain oil composition feedstock is combined with water to form an oil-water mixture and form an oil phase (grain oil product) and an emulsion phase and so that at least a portion of the impurity component can transfer into the emulsion phase, thereby advantageously producing a relatively more pure grain oil product as compared to the grain oil composition feedstock.

The amount of water combined with the grain oil composition feedstock is selected so that the amount of water in the oil-water mixture is from 5-50% based on the total volume of the oil water mixture (v/v). Accordingly, the amount of water can vary, for example, based on the amount of water in the grain oil composition feedstock. In some embodiments, the amount of water in the oil-water mixture is from 10-40% based on the total volume of the oil water mixture (v/v), from 15-35% based on the total volume of the oil water mixture (v/v), from 5-10% based on the total volume of the oil water mixture (v/v), from 8-15% based on the total volume of the oil water mixture (v/v), from 10-25% based on the total volume of the oil water mixture (v/v), from 20-35% based on the total volume of the oil water mixture (v/v), from 25-50% based on the total volume of the oil water mixture (v/v), or even from 3-50% based on the total volume of the oil water mixture (v/v).

The amount of water added can affect how many layers, or phases, form from the oil-water mixture. In some embodiments, the oil-water mixture forms into at least an oil layer/phase and an emulsion layer/phase. In some embodiments, if a relatively large amount of water is combined with the grain oil composition feedstock, the oil-water mixture can form into three layers/phases. Namely, an oil layer, an emulsion layer and a water layer. In some embodiments, the oil-water mixture forms into a top layer, or light, oil phase and a bottom layer, or heavy, emulsion phase.

Water can be obtained from a variety of sources. Nonlimiting examples of water sources include standard tap water, biorefinery distillate, reverse osmosis (RO) reject, RO permeate, de-ionized water, or any other suitable water source. While not being bound by theory, it is believed that with respect to at least some impurities adding water can provide a concentration gradient to facilitate transferring one or more impurities into an emulsion phase so that they can be sequestered from the oil phase. Not wishing to be bound by theory, it is believed that the addition of water may also hydrate one or more impurities that are sequestered in an emulsion phase that may be separated from the oil phase that becomes the grain oil product. The term sequestering as used herein refers to the process wherein contaminants are either directly or indirectly (through binding to water molecules) taken up into the emulsion phase.

Optionally, one or more chelating agents can be added to the water to aid in removal of metals and metal compounds from the grain oil composition feedstock. In some embodiments, the metals are calcium, potassium, magnesium, aluminum, iron, and copper. Metal contaminants, especially iron, can darken oil during other processing steps (e.g. deodorizing of oil), and even small amounts of iron that do not affect the oil's color can reduce stability of refined oil.

Exemplary chelating agents include ethylenediaminetetraacetic acid (EDTA). Commercially available EDTA is sold as VERSENE 100 (Dow Chemical). It is envisioned that any suitable chelating agent could be used.

FIG. 1 is a schematic block diagram illustrating an embodiment 100 of refining a grain oil composition feedstock according to the present disclosure. As shown, a source of grain oil composition feedstock 101 and a source of water 102 are combined in a system 105 to form an emulsion phase and an oil phase.

A wide variety of systems and apparatuses can be used to combine and mix the grain oil composition feedstock 101 and source of water 102. One or more mixing/agitation apparatuses can be combined in series and/or in parallel. Nonlimiting examples include one or more static mixers (e.g., inline static mixers), impeller mixers, pumps, shear mixers, tank recirculation loops, tank mixers (e.g., continuously stirred tank reactors), and combinations thereof (e.g., in series), or other mixers able to disperse the water in the grain oil composition and promote thorough mixing. In some embodiments, a grain oil composition feedstock stream 101 and a water stream 102 can be piped to combine in a common pipe and mix together.

A wide variety of conditions can be used to form an emulsion phase and an oil phase. For example, the oil-water mixture is exposed to a temperature in the range from 0° C. to 50° C. for a time period at least until the oil-water mixture forms at least an oil phase and an emulsion phase. In some embodiments, the oil-water mixture is exposed to a temperature in the range from 5° C. to 35° C., 10° C. to 30° C., 15° C. to 25° C., or even from 20° C. to 30° C. In some embodiments, the oil-water mixture is at a temperature in the range from 0° C. to 5° C., or 0° C. to 10° C. In some embodiments, the oil and water are mixed in a refrigerated vessel that is at 2° C. to 7° C. or from 3° C. to 5° C. temperature for 30 minutes to 5 hours. In some embodiments, oil-water mixture is simply allowed to cool over time to e.g., 10° C. to 40° C.

In some embodiments, the two phases can be cooled (e.g., about 4° C.) in a container so that the heavy phase fraction forms a solid. The liquid light phase fraction can then be easily separated from the heavy phase fraction. Additionally, the corn oil in FIG. 1 may be treated prior to separation. Treatment methods may include degumming, adding a flocculating agent to the corn oil, adding a filter aid to the corn oil, or a combination of these methods. It is recognized that the separation process in FIG. 1 may be suitable for separating other vegetable oils. Also, an as-is distiller's corn oil can be combined with an amount of water prior to separating into a light phase fraction and heavy phase fraction. It was discovered that combining, e.g., 20% w/w of water with as-is distiller's corn oil prior to separation resulting in a heavy phase fraction that was a bright yellow, homogenous emulsion.

FIG. 4 is a schematic process flow diagram illustrating a more detailed embodiment of refining a grain oil composition feedstock according to the present disclosure. As shown in FIG. 4, the grain oil composition feedstock 401 can be relatively hot or cool before it is combined with water to form an emulsion according to the present disclosure. For example, the grain oil composition feedstock 401 can be at a temperature greater than 50° C. (e.g., from 70° C. to 105° C., or even from 70° C. to 95° C.) or less than 0° C. And, for example, if the grain oil composition feedstock is at a temperature above 50° C. (e.g., 90° C.) it may be challenging to the form an emulsion phase. Accordingly, the grain oil composition feedstock 401 can be cooled or heated so that it is at a temperature in the range from 0° C. to 50° C. A variety of heating or cooling techniques can be used. For example, the temperature of the grain oil composition feedstock 401 can be adjusted (heated or cooled) by using a heat exchanger and/or by mixing the grain oil composition feedstock 401 with an appropriate amount of water at an appropriate temperature.

As shown in FIG. 4, the grain oil composition feedstock 401 is pumped via pump 402 through a heat exchanger 405 that utilizes a heat transfer medium 406 (e.g., water) to adjust the temperature of the grain oil composition feedstock 401. Heat exchanger 405 may be a plate and frame heat exchanger, a shell and tube heat exchanger, or some other heat exchanger that is suitable for cooling or heating a grain oil stream. The heat exchange fluid 406 that is used to transfer heat may be process water at a biorefinery. If colder temperatures are desired, the heat exchange fluid may be chilled glycol or a different heat exchange fluid. As shown, water 408 is combined with the temperature adjusted grain oil composition feedstock 407, e.g., via mixing device 410. The temperature of the grain oil composition feedstock 401 (if heat exchanger 405 is omitted) or the temperature adjusted grain oil composition feedstock 407 may be adjusted (heated or cooled) by the temperature of the water 408 that is combined with it in the mixing device 410 (e.g., an inline static mixer) to form an oil-water mixture 411 at a temperature in the range from 0° C. to 50° C. In some embodiments, the temperature of the oil-water mixture 411 is from 0° C. to 50° C. by adding water 408 that is at a temperature of 10° C. to 40° C. In some embodiments the temperature adjusted grain oil composition feedstock 407 may be cooled (e.g. to less than 30° C.) before adding the water 408. In still other embodiments, the temperature of the oil-water mixture 411 once formed may be further adjusted (e.g., cooled). Although not shown, water stream 408 or an additional water stream could be combined with the grain oil composition feedstock 401 and/or the temperature adjusted grain oil composition feedstock 407 directly in mixing tank 415.

As shown, the oil-water mixture can be further agitated to facilitate forming an oil phase and an emulsion phase so that at least a portion of the impurity component is sequestered in the emulsion phase. As shown in the illustrative example of FIG. 4, the oil-water mixture 411 is fed into tank mixer 415, wherein it is mixed using mixing impellers and is recirculated via pump 416 and recirculation line 417.

Forming an oil phase and emulsion phase as described herein can be performed in a continuous or batch manner. In some embodiments, when the process is carried out as a batch process, the grain oil composition feedstock and water can be introduced sequentially or simultaneously and in any order. If the oil-water mixture is agitated and then allowed to settle it will form layers. In some embodiments when the process is carried out as a continuous process, inline static mixer 410 can instead be an intersection of piping where grain oil composition feedstock and water are introduced simultaneously and then subsequently mixed by a mixing device, such as a static mixer.

The oil-water mixture can be mixed for a time period to form the oil phase and emulsion phase and permit at least a portion of the impurity component to be sequestered in the emulsion phase. Mixing parameters are selected according to the mechanical design of the mixer. Mixing may be performed from a period of fractions of a second to hours, e.g., from 5 minutes to 5 hours. Mixing may occur in a continuous flow mixing vessel. If so, adjusting the volume of the continuous flow reactor will adjust the mean residence time of the oil-water mixture in the reactor, thereby controlling the length of the mixing period; e.g. for a given flow through rate, a larger reactor vessel will provide a longer mean residence period.

An example of combining a grain oil composition feedstock with water to form an oil-water mixture and form an oil phase (grain oil product) and an emulsion phase so that at least a portion of the impurity component can transfer into the emulsion phase is described in U.S. Patent Publication 2019/0376002 (Urban et al.), wherein the entirety of said patent publication is incorporated herein by reference.

Recovering the Oil Phase From the Emulsion Phase

As shown in FIG. 1, after forming the emulsion phase and the oil phase, at least a portion of the oil phase can be separated and recovered 110 from the emulsion phase 111 to form a grain oil product 112. The emulsion phase 111 can also be referred to as a by-product of the grain oil composition feedstock 101. A wide variety of systems and devices can be used to separate at least a portion of the emulsion phase from the oil phase. Because the oil phase has a bulk density that is less than the bulk density of the emulsion phase, separation techniques based on density differences can be used. In some embodiments, separation is accomplished by allowing gravity phase separation to occur over time using a settling tank and/or a cooled settling tank, followed by decanting the oil phase layer. In some embodiments, separation is accomplished more quickly by centrifugation. These and other methods may be combined. Centrifugation can be by, for example, a decanter centrifuge, a disk stack centrifuge, a cooled disk stack centrifuge, a screen centrifuge, hydrocyclone or a combination thereof. The speed or amount of centrifugal force applied can depend on various factors such as sample size and may be adjusted appropriately depending on such factors. For example, centrifugation may be carried out at 4,200 rpm. In some embodiments, centrifugation is carried out at 4,200 rpm, for 20 minutes and at 27° C. Nonlimiting examples of other apparatuses that can be used to separate an emulsion phase from an oil phase include a filter press, a rotary drum filter, or some other apparatus that is suitable to separate a liquid stream based on density differences.

Referring to the illustrative example of FIG. 4, the oil water mixture 418 is passed through a centrifuge 420 to separate emulsion (heavy phase) 423 from the oil phase (light phase) 424.

After isolating a grain oil product (light phase fraction) and an emulsion phase (heavy phase fraction), the grain oil product and/or the emulsion phase can be filtered to remove solid particles and/or waxy particles. Waxy particles refer to particles that may settle out at a given temperature (e.g., 21° C.). As shown in FIG. 4, the oil phase 424 can be transferred to a surge tank (collection tank) 421. The pressure of the light phase 424 leaving the centrifuge 420 may not be high enough to pass through downstream equipment. Surge tank 421 and pump 422 facilitate pumping light phase 424 downstream. Also, surge tank 421 and pump 422 can help provide a consistent flow rate, for example, when the flow of light phase 424 from centrifuge 420 experiences fluctuations. As shown, light phase 424 is pumped through one or more filter apparatuses 430 in series or parallel to remove at least a portion of solids, waxy particles, soaps, metals, and combinations thereof from the oil phase 424 and form a final grain oil product 435. Non-limiting examples of filter apparatuses include a filter press, a cylindrical cartridge filter, a pleated cartridge filter, a sock filter, and combinations thereof. In some embodiments, a filter's nominal micrometer rating may be 1, 2, 3, 4, 5, 6, 7, 8, 9, or even 10 microns.

As shown, depending on the end use, any solids, waxy particles, soaps, metals, and combinations thereof that are separated from the oil phase 424 can be transferred via stream 431 and combined with the emulsion phase 423 in one or more tanks 425 and form an emulsion product 426.

According to the present disclosure, an amount of the impurity component in the emulsion phase is greater than an amount of the impurity component in the grain oil product. In some embodiments, at least 50 percent by weight of the impurity component in the grain oil feedstock composition is present in the emulsion phase, at least 60 percent by weight of the impurity component in the grain oil feedstock composition is present in the emulsion phase, at least 70 percent by weight of the impurity component in the grain oil feedstock composition is present in the emulsion phase, at least 80 percent by weight of the impurity component in the grain oil feedstock composition is present in the emulsion phase, at least 90 percent by weight of the impurity component in the grain oil feedstock composition is present in the emulsion phase, or even at least 95 percent by weight of the impurity component in the grain oil feedstock composition is present in the emulsion phase.

As mentioned, the oil phase can be a final grain oil product or can be further treated (see below) to become a final grain oil product. Because of the water refining process described herein, the grain oil product can be referred to as “refined” because it can have a relatively higher triglyceride content and relatively less impurity component than was present in the grain oil composition feedstock and/or that is present in the emulsion phase (and/or optional aqueous, third phase/layer). In some embodiments, the grain oil product passes visual inspection as being clear and bright after 48 hr. incubation at 0° C. In some embodiments, the oil yield achieved by the described refining process is at least 60 percent of the starting grain oil feedstock composition, at least 70 percent of the starting grain oil feedstock composition, at least 80 percent of the starting grain oil feedstock composition, or even at least 90 percent of the starting grain oil feedstock composition.

As mentioned, the grain oil product includes at least a triglyceride component having one or more triglycerides. The amount of the triglyceride component in the grain oil product can depend on, e.g., the amount present in the grain oil feedstock composition. In some embodiments, the triglyceride component can be present in an amount of at least 70 percent by weight of the total grain oil product, at least 80 percent by weight of the total grain oil product, at least 90 percent by weight of the total grain oil product or even at least 95 percent by weight of the total grain oil product. In some embodiments, the triglyceride component can be present in an amount from 70 to 99 percent by weight of the total grain oil product, from 75 to 95 percent by weight of the total grain oil product, from 80 to 95 percent by weight of the total grain oil product, or even from 85 to 95 percent by weight of the total grain oil product. Triglycerides can be determined by test method AOCS Cd 11d-96.

In some embodiments, the grain oil product includes a diglyceride component having one or more diglycerides. The amount of the diglyceride component in the grain oil product can depend on, e.g., the amount present in the grain oil feedstock composition. In some embodiments, the diglyceride component can be present in an amount of 30 percent or less by weight of the total grain oil product, 20 percent or less by weight of the total grain oil product, 10 percent or less by weight of the total grain oil product, or even 5 percent or less by weight of the total grain oil product. In some embodiments, the diglyceride component can be present in an amount from 1 to 20 percent by weight of the total grain oil product, from 1 to 15 percent by weight of the total grain oil product, from 1 to 10 percent by weight of the total grain oil product, or even from 1 to 5 percent by weight of the total grain oil product. Diglycerides can be determined by test method AOCS Cd 11d-96.

In some embodiments, the grain oil product includes a monoglyceride component having one or more monoglycerides. The amount of the monoglyceride component in the grain oil product can depend on, e.g., the amount present in the grain oil feedstock composition. In some embodiments, the monoglyceride component can be present in an amount of 20 percent or less by weight of the total grain oil product, 15 percent or less by weight of the total grain oil product, 10 percent or less by weight of the total grain oil product, or even 5 percent or less by weight of the total grain oil product. In some embodiments, the monoglyceride component can be present in an amount from 1 to 15 percent by weight of the total grain oil product, from 1 to 10 percent by weight of the total grain oil product, from 1 to 5 percent by weight of the total grain oil product, or even from 0.1 to 5 percent by weight of the total grain oil product. Monoglycerides can be determined by test method AOCS Cd 11d-96.

The moisture content in the grain oil product can depend on, e.g., the moisture content present in the grain oil feedstock composition. In some embodiments, the grain oil product includes a moisture content of 20 percent or less by weight of the total grain oil product, 10 percent or less by weight of the total grain oil product, 5 percent or less by weight of the total grain oil product, 1 percent or less by weight of the total grain oil product, or even 0.5 percent or less by weight of the total grain oil product. In some embodiments, the moisture content can be from 0.01 to 5 percent by weight of the total grain oil product, from 0.01 to 1 percent by weight of the total grain oil product, from 0.01 to 0.5 percent by weight of the total grain oil product, or even from 0.1 to 0.5 percent by weight of the total grain oil product. Moisture content can be determined by a Karl Fischer titration (e.g., following ASTM E1064-12 or AOCS 2e-84).

In some embodiments, the grain oil product may include some to the impurity component that was present in the grain oil composition feedstock. As discussed herein, in some embodiments it is desirable to perform the refining process described herein to sequester as much of the impurity component in the grain oil composition feedstock in the emulsion phase as possible. In some embodiments, the grain oil product includes an impurity component having one or more elements chosen from aluminum, arsenic, cadmium, calcium, chlorides, chromium, copper, iron, lead, magnesium, manganese, mercury, nitrogen, nickel, phosphorus, potassium, silicon, sodium, sulfur, vanadium, zinc, and combinations thereof. In some embodiments, the impurity component includes at least one element chosen from calcium, phosphorus, potassium, sodium, and combinations thereof. Metals can be determined by test method AOCS Ca 17-01.

Phosphorus can be determined by test method AOCS Ca 20-99. Sulfur can be determined by test method ASTM D4951.

In some embodiments, the grain oil product includes the element component in an amount of 500 parts per million (ppm) or less based on the total grain oil product, 200 ppm or less based on the total grain oil product, 100 ppm or less based on the total grain oil product, 50 ppm or less based on the total grain oil product, or even 25 ppm or less based on the total grain oil product. In some embodiments, the grain oil product includes the element component in an amount from about 0-100 ppm, from 0-50 ppm, 0-10 ppm, 0-5 ppm, 5-20 ppm, 10-30 ppm, 2550 ppm, 35-60 ppm, 45-75 ppm, 50-75 ppm, or even 75-100 ppm.

In some embodiments, at least a portion (e.g., including substantially all) of the element component is present as soap, which is a salt of the element and a fatty acid such as sodium oleate, magnesium stearate, combinations of these, and the like. In some embodiments, grain oil product includes a soap component in an amount from 0 to 500 ppm, from 0 to 100 ppm, from 0 to 50 ppm, or even from 0 to 10 ppm. Soap content can be determined by test method AOCS Cc17-95.

In some embodiments, the grain oil product contains no detectable phospholipid. For example, any phospholipid that may have been inherently present in the raw grain material may have been removed in an upstream process.

A grain oil product can also include a fatty acid alkyl ester (FAAE) component including one or more fatty acid alkyl esters such as fatty acid ethyl ester (FAEE), which is an esterified (not free) fatty acid. Nonlimiting examples of fatty acid ethyl esters include one or more of ethyl linoleate, ethyl linolenate, ethyl oleate, ethyl palmitate, and ethyl stearate. In some embodiments, the amount of the one or more fatty acid alkyl esters is in the range from 0 to 30 percent by weight based on the total weight of the grain oil product, from 0.5 to 20 percent by weight based on the total weight of the grain oil product, or from 1 to 15 percent by weight based on the total weight of the grain oil product.

A grain oil product can also include a free fatty acid component including one or more free fatty acids. In some embodiments, the amount of the one or more free fatty acids is the same as after treatment with esterase enzyme component as described above.

The emulsion phase can be a final emulsion product or can be further treated (see below) to become a final emulsion product. Because of the water refining process described herein, the emulsion phase can have relatively more impurity component than was present in the grain oil composition feedstock and/or that is present in the grain oil product.

The emulsion phase may include at least a triglyceride component having one or more triglycerides. The amount of the triglyceride component in the emulsion phase can depend on, e.g., the amount present in the grain oil feedstock composition and the yield in the grain oil product. In some embodiments, the triglyceride component can be present in an amount of 80 percent or less by weight of the total emulsion phase, 70 percent or less by weight of the total emulsion phase, 60 percent or less by weight of the total emulsion phase, or even 50 percent or less by weight of the total emulsion phase. In some embodiments, the triglyceride component can be present in an amount from 0 to 70 percent by weight of the total emulsion phase, from 5 to 50 percent by weight of the total emulsion phase, from 10 to 40 percent by weight of the total emulsion phase, or even from 15 to 30 percent by weight of the total emulsion phase. Triglycerides can be determined by test method AOCS Cd 11d-96.

In some embodiments, the emulsion phase includes a diglyceride component having one or more diglycerides. The amount of the diglyceride component in the emulsion phase can depend on, e.g., the amount present in the grain oil feedstock composition. In some embodiments, the diglyceride component can be present in an amount of 10 percent or less by weight of the total emulsion phase, 5 percent or less by weight of the total emulsion phase, or even 1 percent or less by weight of the total emulsion phase. Diglycerides can be determined by test method AOCS Cd 11d-96. In some embodiments, the emulsion phase includes a monoglyceride component having one or more monoglycerides. The amount of the monoglyceride component in the emulsion phase can depend on, e.g., the amount present in the grain oil feedstock composition. In some embodiments, the monoglyceride component can be present in an amount of 10 percent or less by weight of the total emulsion phase, or even 1 percent or less by weight of the total emulsion phase. Monoglycerides can be determined by test method AOCS Cd 11d-96. The moisture content in the emulsion phase can depend on, e.g., the moisture content present in the grain oil feedstock composition and water added to the grain oil composition feedstock. In some embodiments, the emulsion phase includes a moisture content of 20 percent or more by weight of the total emulsion phase, 30 percent or more by weight of the total emulsion phase, 40 percent or more by weight of the total emulsion phase, 50 percent or more by weight of the total emulsion phase, or even 60 percent or more by weight of the total emulsion phase. In some embodiments, the moisture content can be from 20 to 70 percent by weight of the total emulsion phase, from 30 to 65 percent by weight of the total emulsion phase, or even from 35 to 65 percent by weight of the total emulsion phase. Moisture content can be determined by a Karl Fischer titration (e.g., following ASTM E1064-12 or AOCS 2e-84).

As discussed herein, it can be desirable to perform the refining process described herein to sequester as much of the impurity component in the grain oil composition feedstock in the emulsion phase as possible. In some embodiments, the emulsion phase includes an impurity component having one or more elements chosen from aluminum, arsenic, cadmium, calcium, chlorides, chromium, copper, iron, lead, magnesium, manganese, mercury, nitrogen, nickel, phosphorus, potassium, silicon, sodium, sulfur, vanadium, zinc, and combinations thereof. In some embodiments, the impurity component includes at least one element chosen from calcium, phosphorus, potassium, sodium, and combinations thereof. Metals can be determined by test method AOCS Ca 17-01. Phosphorus can be determined by test method AOCS Ca 20-99.

Sulfur can be determined by test method ASTM D4951.

In some embodiments, the emulsion phase includes the element component in an amount of 100 parts per million (ppm) or more based on the total emulsion phase, 200 ppm or more based on the total emulsion phase, 500 ppm or more based on the total emulsion phase, 1000 ppm or more based on the total emulsion phase, or even 10,000 ppm or more based on the total emulsion phase. In some embodiments, the element component can be from 5 to 50,000 ppm based on the total emulsion phase, from 100 to 10,000 ppm based on the total emulsion phase, or even from 1000 to 40,000 ppm based on the total emulsion phase.

In some embodiments, at least a portion (e.g., including substantially all) of the element component is present as soap, which is a salt of the element and a fatty acid such as sodium oleate, magnesium stearate, combinations of these, and the like. In some embodiments, emulsion phase includes a soap component in an amount from 50 to 100,000 ppm, from 100 to 50,000 ppm, or even from 500 to 20,000 ppm. Soap content can be determined by test method AOCS Cc17-95.

In some embodiments, the emulsion phase contains no detectable phospholipid. For example, any phospholipid that may have been inherently present in the raw grain material may have been removed in an upstream process.

An emulsion phase can also include a fatty acid alkyl ester (FAAE) component including one or more fatty acid alkyl esters such as fatty acid ethyl ester (FAEE), which is an esterified (not free) fatty acid. Nonlimiting examples of fatty acid ethyl esters include one or more of ethyl linoleate, ethyl linolenate, ethyl oleate, ethyl palmitate, and ethyl stearate. In some embodiments, the amount of the one or more fatty acid alkyl esters is in the range from 0 to 30 percent by weight based on the total weight of the emulsion phase, from 0.5 to 20 percent by weight based on the total weight of the emulsion phase, or from 1 to 15 percent by weight based on the total weight of the emulsion phase.

An emulsion phase can also include a free fatty acid component including one or more free fatty acids. In some embodiments, the amount of the one or more free fatty acids is in the range from 0 to 30 percent by weight based on the total weight of the emulsion phase, from 0.5 to 20 percent by weight based on the total weight of the emulsion phase, or from 1 to 15 percent by weight based on the total weight of the emulsion phase. Free fatty acid can be determined by test method AOCS Ca 5a-40.

The emulsion phase may be used as-is, dried to an anhydrous oily emulsion product, or dried and de-oiled to provide a solid emulsion product (e.g., solvent extracted to yield a de-oiled powder). Where the emulsion phase is dried, e.g. by evaporation in an evaporator, the removed water can be recycled for re-use in the refining process. Water recycling results in substantially no discharge water. In embodiments, the water is recycled without the need for treatment. In some embodiments, drying is performed using a wiped film evaporator to minimize heat degradation of the product if degradation is to be minimized.

Drying of the emulsion phase results in a concentrated emulsion product, which may include triglycerides, diglycerides, monoglycerides, free fatty acids, and fatty acid soaps. De-oiling the emulsion product further concentrates the emulsion product. In some embodiments, a dried and de-oiled emulsion comprises primarily soaps in powder form.

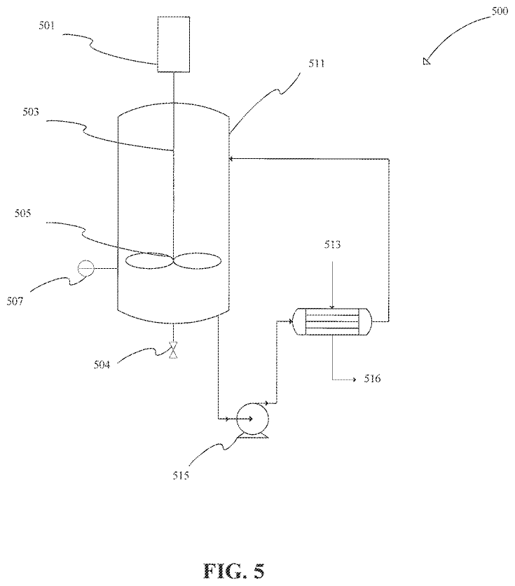

FIG. 5 illustrates an energy saving embodiment of a system 500 for removing water from the emulsion by heating the emulsion to form an at least partially dehydrated emulsion layer/phase and an aqueous layer/phase. As shown in FIG. 5, an emulsion can be provided in an insulated tank 511 and mixed using a mixing system that includes mixer motor 501, mixer shaft 503, and mixer blades 505. The temperature of the emulsion can be adjusted, e.g., from 80° C. to 100° C. by, e.g., circulating the emulsion through a heat exchanger 513 via a pump 515. Hot water or steam is supplied to the heat exchanger to heat the emulsion. Steam condensate 516 can be directed as desired. The temperature can be monitored using temperature sensor 507. The emulsion can be kept at from 80° C. to 100° C. and mixed for a time period to cause a desired separation of the emulsion into an aqueous phase and a dehydrated emulsion phase. In some embodiments, the emulsion can be mixed for a time period of 5 minutes to 2 hours. After mixing, the contents can settle for a time period (e.g., from 15 minutes to an hour) to allow the dehydrated emulsion phase and aqueous phase to form. The aqueous phase may be drained via drain valve 504. Alternatively, the emulsion phase can be heated without mixing to form the dehydrated emulsion phase and aqueous phase, but it may take longer. As yet another alternative, the emulsion phase may be heated to evaporate moisture but that may require more energy.

In some embodiments, a dehydrated emulsion product can have a moisture content of 1% or less based on the total weight of the dehydrated emulsion product.

In some embodiments, the dehydrated emulsion product includes a moisture content of 20 percent or less by weight of the total dehydrated emulsion product, 10 percent or less by weight of the total dehydrated emulsion product, 5 percent or less by weight of the total dehydrated emulsion product, 1 percent or less by weight of the total dehydrated emulsion product, or even 0.5 percent or less by weight of the total dehydrated emulsion product. In some embodiments, the moisture content can be from 0.01 to 5 percent by weight of the total dehydrated emulsion product, from 0.01 to 1 percent by weight of the total dehydrated emulsion product, from 0.01 to 0.5 percent by weight of the total dehydrated emulsion product, or even from 0.1 to 0.5 percent by weight of the total dehydrated emulsion product. Moisture content can be determined by a Karl Fischer titration (e.g., following ASTM E1064-12 or AOCS 2e-84).

In some embodiments, a dehydrated emulsion product can have at least 100 ppm of an element component, wherein the element component comprises at least one element chosen from calcium, phosphorus, potassium, sodium, and combinations thereof.

Optionally, a grain oil composition (e.g., grain oil composition feedstock and/or oil phase (grain oil product)) and/or a byproduct of a grain oil composition (e.g., emulsion phase and/or dehydrated emulsion phase) can be further treated by one or more processes. Non-limiting examples of such processes include one or more filtering, bleaching, deodorizing and/or homogenizing (to reduce separation) to improve its usefulness in various applications.

Bleaching

As shown in FIG. 1, in some embodiments, a grain oil product can be treated using one or more bleaching processes 114 to change the color of the corn oil and form a “bleached” grain oil product 116. In some embodiments, it can be desirable to change the color of the grain oil product via a bleaching process so that the bleached grain oil product has a color that is compatible with its end use. For example, as mentioned below, a corn oil according to the present disclosure can be used as a functional replacement for mineral oil. Because mineral oil can be a relatively clear or lighter color than some grain oils, it can be desirable to bleach a grain oil according to the present disclosure so that it resembles mineral oil in color.

A grain oil product can include one or more pigments that impart color to the grain oil product. Pigments can include naturally present pigments such as organic pigments. Non-limiting examples of pigments include tocopherols, tocotrienols, and/or carotenoids. Non-limiting examples of carotenoids include alpha-carotene, beta-carotene, and xanthophyll. Non-limiting examples of xanthophyll include lutein and zeaxanthin. In some embodiments, after bleaching the concentration of tocopherols can be less than 1000 ppm, less than 500 ppm, less than 100 ppm, or even less than 50 ppm. In some embodiments, after bleaching the concentration of tocotrienols can be less than 500 ppm, less than 100 ppm, less than 50 ppm, or even less than 25 ppm. In some embodiments, after bleaching the concentration of lutein can be less than 200 ppm, less than 25 ppm, less than 10 ppm, or even less than 5 ppm. In some embodiments, after bleaching the concentration of zeaxanthin can be less than 200 ppm, less than 25 ppm, less than 10 ppm, or even less than 5 ppm.

A variety of bleaching processes for changing the color of grain oil can be used. Non-limiting bleaching processes include one or more of applying heat to grain oil, contacting grain oil with oxidizing gas, extracting one or more pigments in a grain oil with supercritical fluid, extracting one or more pigments in a grain oil with ultrasound-assisted extraction (UAE), extracting one or more pigments in a grain oil with pressurized liquid extraction (PLE), contacting grain oil with bleaching agents (e.g., hydrogen peroxide treatment, benzyl peroxide treatment, activated charcoal plus hydrogen peroxide treatment, etc.), contacting grain oil with adsorbents, and combinations thereof.

In some embodiments, a grain oil product can be bleached by heating it to a temperature from 100° C. to 180° C., from 105° C. to 170° C., from 110° C. to 160° C., from 115° C. to 150° C., or even from 115° C. to 135° C. In some embodiments, the rate of color change of a grain oil product can be relatively higher at a relatively higher temperature and relatively lower at a relatively lower temperature. Also, in some embodiments, the final color of a grain oil product may be relatively lighter for a grain oil product that was heated at a relatively lower temperature as compared to the final color of a grain oil product that was heated at a relatively higher temperature.

A grain oil product can be bleached by heating according to the present disclosure with or without contacting the grain oil product with an oxidizing gas. In some embodiments, if an oxidizing gas is not used, a relatively higher temperature can be used to bleach the grain oil product as compared to if an oxidizing gas was used.

A variety of oxidizing gases can be used according to the present disclosure to bleach a grain oil product. Non-limiting examples of an oxidizing gas include air, oxygen, ozone and combinations thereof. Can be compressed or atmospheric pressure.

An oxidizing gas can be caused to contact a grain oil product in a variety of ways to oxidize one or more pigments present in the grain oil product to change the color of the grain oil product and form a bleached grain oil product. For example, an oxidizing gas can be introduced into the headspace of a closed vessel so that the oxidizing gas diffuses into the grain oil product.

As another example, an oxidizing gas can be sparged into the grain oil product so that the oxidizing gas bubbles up and through the grain oil product and oxidizing gas transfers into the grain oil product. As another example, falling film techniques can be used to contact a grain oil product with an oxidizing gas to bleach the grain oil product as desired. For example, a grain oil product can be caused to flow over a weir in an atmosphere of oxidizing gas.

Optionally, the grain oil product can be agitated or mixed to facilitate transferring oxidizing gas into and throughout the grain oil product so as to achieve the desired bleaching results. For example, a continuous stirred tank reactor (CSTR) can be used to agitate or mix the grain oil product. The speed of the stirring mechanism (rpms) can be adjusted based on a variety of factors such as tank size, oil viscosity, and the like.

A flowrate of an oxidizing gas can be selected for introduction into a volume of grain oil product to contact the grain oil product in a variety of ways to oxidize one or more pigments present in the grain oil product to change the color of the grain oil product and form a bleached grain oil product. In some embodiments, a ratio of volumetric flow of oxidizing gas to volume of oil grain oil product can be from 0.5 to 10 lpm of oxidizing gas per liter of oil; from 0.75 to 9 lpm of oxidizing gas per liter of oil; from 1 to 8 lpm of oxidizing gas per liter of oil; or even 1.5 to 7 lpm of oxidizing gas per liter of oil.

A grain oil product can be exposed to one or more conditions (e.g., heat and air) for a time period to oxidize one or more pigments present in the grain oil product to change the color of the grain oil product and form a bleached grain oil product. The time period selected can depend on a variety of factors such as temperature and ratio of oxidizing gas volumetric flowrate to volume of grain oil product, and the like. In some embodiments, a grain oil product can be exposed to one or more conditions (e.g., heat and air) for a time period from 30 minutes to 6 hours, or even 45 minutes to 5 hours.

As described above, treating a grain oil with an esterase enzyme component can reduce or prevent the formation of metal soap. Because metal soap can have antioxidant properties, a grain oil product according to the present disclosure having relatively less metal soap can have a relatively low oxidative stability index (OSI). Bleaching a grain oil product having a low OSI with an oxidizing gas (with or without heating) can be performed at relatively less severe conditions, which can avoid an undue increase in viscosity if desired. In some embodiments, prior to bleaching with an oxidizing gas, a grain oil product can have an OSI of 7 hours or less, 6 hours or less, 5 hours or less, 4 hours or less, 3 hours or less, 2 hours or less 1.5 hours or less, 1 hour or less, or even 45 minutes or less according to AOCS Official Method Cd 12b-92-Reapproved 1997. As discussed below, a grain oil product can be filtered prior to bleaching, which can reduce any metal soap content (and OSI) even further.

A grain oil product can be bleached according to a batch process or a continuous process.

In some embodiments, a grain oil product can be bleached according to a batch process and a continuous process in series. Non-limiting examples are described herein below with respect to FIGS. 10-12. For illustration purposes, non-limiting examples shown in each of FIGS. 10-12 are described in the context of contacting a grain oil with oxidizing gas while the grain oil is at an elevated temperature, which can be an economical process that provides desirable color results according to the present disclosure.

Referring to FIG. 10, a batch process 1000 can include a vessel 1010 that includes grain oil product 1030 contained therein while it is heated via a heat source 1040. While grain oil product 1030 is in vessel 1010 at an elevated temperature it can be contacted with on oxidizing gas 1020 to oxidize one or more pigments and change the color of grain oil product as desired.

In some embodiments, the grain oil product can be mixed with a stirring mechanism 1005 while it is being heated and exposed to an oxidizing gas.

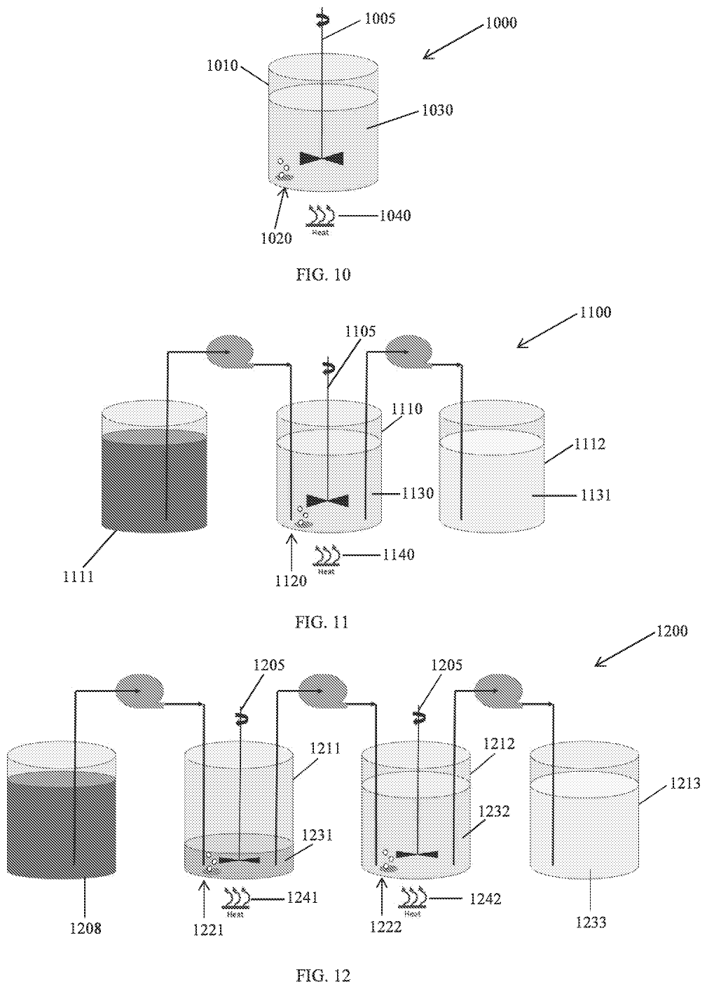

Referring to FIG. 11, a continuous flow process 1100 can include a vessel 1110 that includes grain oil product 1130 contained therein while it is heated via a heat source 1140. While grain oil product 1130 is in vessel 1110 at an elevated temperature it can be contacted with an oxidizing gas 1120 that is, as shown in FIG. 11, sparged into the grain oil product 1130 at a flowrate to oxidize one or more pigments and change the color of grain oil product as desired. In some embodiments, the grain oil product can be mixed with a stirring mechanism 1105 while it is being heated and exposed to an oxidizing gas.

A grain oil product can be exposed to conditions of temperature and oxidizing gas as described above with respect to FIG. 10. The process 1100 is a continuous process, but the residence time of the grain oil product in vessel 1110 can be selected as desired to achieve the desired bleaching effect. In some embodiments, the resident time is from 30 minutes to 2 hours at a temperature from 110 to 130° C.

As shown in FIG. 11, continuous process 1100 is a single-stage process and is illustrated by a grain oil product 1130 being continuously fed from vessel 1111 and bleached grain oil product 1131 being continuously discharged into vessel 1112.

Referring to FIG. 12, a continuous flow process 1200 can include a first vessel 1211 that includes grain oil product 1231 contained therein while it is heated via a heat source 1241. While grain oil product 1231 is in first vessel 1211 at a first elevated temperature for a first residence time period it can be contacted with an oxidizing gas 1221 that is, as shown, sparged into the grain oil product 1231 at a flowrate to oxidize one or more pigments and change the color of grain oil product as desired and form a partially bleached grain oil product 1232 that is fed into second vessel 1212. While partially bleached grain oil product 1232 is in second vessel 1212 at a second elevated temperature for a second residence time period it can be contacted with an oxidizing gas 1222 that is, as shown, sparged into the partially bleached grain oil product 1232 at a flowrate to oxidize one or more pigments and change the color of grain oil product as desired and form a target bleached grain oil product 1233 that is fed into vessel 1213.

In some embodiments, a stirring mechanism 1205 can mix the contents of first and second vessels 1211 and 1212 while the contents are being heated and exposed to an oxidizing gas.

A multi-staged system such as that shown in FIG. 12 can facilitate reducing the overall time to bleach an oil by appropriately selecting temperature and residence time in each vessel. In some embodiments, the first residence time can be from 30 to 90 minutes (or even 40 to 70 minutes) and the first temperature can be from 150° C. to 170° C. while the second residence time can be from 1 to 3 hours (or even 1.5 to 2.5 hours) and the second temperature can be from 110° C. to 130° C.

As shown in FIG. 12, a grain oil product 1231 is continuously fed from vessel 1208 and bleached grain oil product 1233 is continuously discharged into vessel 1213. An example of a hydrogen peroxide protocol includes mixing a grain oil composition and/or a byproduct of a grain oil composition with 10 vol % of 30% hydrogen peroxide. The mixture can be heated with vigorous stirring to a temperature that causes the water to boil. The mixture can be boiled until a temperature of greater than 130° C. is achieved and all boiling has stopped. The amount of peroxide can be varied, which can result in varying degrees of color change.

An example of an activated charcoal plus hydrogen peroxide includes mixing a grain oil composition and/or a byproduct of a grain oil composition mixed with 2.5 mass % activated charcoal and heating the mixture to 85° C. for 1 hour while mixing. The mixture can be filtered while hot to remove the charcoal. The treated oil can then be combined with 10 vol % of 30% hydrogen peroxide. The mixture can be heated with vigorous stirring to a temperature that causes the water to boil. The mixture can be boiled until a temperature of greater than 130° C. is achieved and all boiling has stopped. The amount of peroxide can be varied, which can result in varying degrees of color change.

An example of air/heat treatment protocol includes sparging a grain oil composition and/or a byproduct of a grain oil composition with compressed air (e.g., at a flowrate of 1 L/min) while heating to a temperature of about 190° C. for a sufficient time period (e.g., about an hour).

An example of extracting one or more pigments using one or more supercritical fluids is described in U.S. Pub. No. 2008/0146851 (Schonemann et al.), wherein the entirety of said publication is incorporated herein by reference.

Filtering

Optionally, a bleaching process can incorporate filtering before, during, or after a bleaching treatment. In some embodiments, a grain oil product can be filtered at least prior to bleaching and/or during bleaching.

In some embodiments, a grain oil product can be filtered using a filter having openings with a size of 5 microns or less, 4 microns or less, 3 microns or less, 2 microns or less, or even 1 micron or less (e.g., a filter having openings from 0.5 to 3 microns).

Grain Oil Products

Embodiments of the present disclosure include grain oil products such as bleached grain oil products. A bleached grain oil product can have a reduced concentration of one or more pigments (e.g., because they have been oxidized) and a change in color.

In some embodiments, a bleached grain oil product can have a viscosity that has not changed to an undue degree because of, e.g., a reduced content of antioxidants (discussed above) prior to bleaching with an oxidizing gas. In some embodiments, a bleached grain oil product can have a Brookfield viscosity in the range from 20 to less than 40 centiPoise (cP) when measured at 40° C. and #SC 4-18 spindle.

In some embodiments, bleaching can be performed under conditions to produce a desired color change in a grain oil product. In some embodiments, prior to bleaching, a grain oil product has a red color value greater than 10, greater than 15, or even greater than 20 on the LOVIBOND RYBN color scale. Bleaching can reduce the red color value to less than 5, less than 4, or even greater than 3 on the LOVIBOND RYBN color scale. In some embodiments, prior to bleaching, a grain oil product has a yellow color value greater than 50, greater than 60, or even greater than 70 on the LOVIBOND RYBN color scale. Bleaching can reduce the yellow color value to less than 30, less than 20, or even greater than 15 on the LOVIBOND RYBN color scale. The LOVIBOND RYBN color scale values are measured according to AOCS Cc 13J-97 using either a 5.25 inch cell or one inch cell.

In one embodiment, a method of bleaching a grain oil product to provide a bleached grain oil product comprises: providing a grain oil product comprising one or more pigments, wherein the grain oil product has an oxidative stability value of 7 hours or less according to AOCS Official Method Cd 12b-92-Reapproved 1997; and contacting the grain oil product with an oxidizing gas under conditions to form a bleached grain oil product. In other embodiments of such methods, the grain oil product has a color value chosen from a red color value greater than 15 on the LOVIBOND RYBN color scale according to AOCS Cc 13J-97, a yellow color value greater than 70 on the LOVIBOND RYBN color scale according to AOCS Cc 13J-97, and combinations thereof, and wherein the bleached grain oil product has a color value chosen from a red color value less than 5 on the LOVIBOND RYBN color scale according to AOCS Cc 13J-97, a yellow color value less than 30 on the LOVIBOND RYBN color scale according to AOCS Cc 13J-97, and combinations thereof.

Using a grain oil composition and/or a byproduct of a grain oil composition

A grain oil composition such as grain oil product or bleached grain oil product produced by a method of refining according to the present disclosure can be used in a wide variety of applications. Such exemplary applications include the areas of oleochemicals, feed (e.g., animal feed) as well as oils suitable for human consumption, an anti-foam agent, and a carrier (e.g., a bio-based mineral oil replacement). In some embodiments, the grain oil composition is a valuable diesel fuel feedstock such as for biodiesel, renewable diesel, low sulfur fuel oil, and co-processing with hydrocarbon stocks. The refined oil can be more compatible with diesel processes than is other distillers oil, e.g. distillers corn oil, because it is less likely to poison catalysts, e.g., due to reduced metal content in the refined oil.

In some embodiments, the grain oil product can be used for asphalt modification, rubber modification, and as a lubricant.

In some embodiments, the grain oil product can be used as a nutrition source. For example, the refined oil may be used in animal and human food formulations. In some embodiments, the refined oil may be used for in pharmaceutical preparations.

A by-product of a grain oil composition produced as a result of refining according to the present disclosure (e.g., an emulsion phase and/or a dehydrated emulsion) can be used for a variety of purposes as well. For example, it has been found that the emulsion and emulsion products behave much like lecithin. The emulsion and emulsion products are believed to be useful, like lecithin, in a variety of applications and perform an array of valuable functions. In edible compositions, they may contribute nutritional value and also can act as an emulsifying agent, surface active agent, anti-spattering-agent, or stabilizing agent. They may be used in technical applications as an anti-foam agent, dispersing agent, wetting agent, stabilizing agent, anti-knock compound, mold release and antioxidant. In cosmetics and pharmaceuticals, they may be used as stabilizer, emollient, emulsifier, wetting agent, softening agent, carrier, and penetration enhancer.

Using a grain oil composition and/or by-product of a grain oil composition to control foam in foamable compositions

A grain oil composition and/or by-product of a grain oil composition as described herein can be used in the context of an anti-foam to help control (prevent or reduce) foaming in a variety of foamable compositions. “Anti-foam” and “defoamer” are used interchangeably herein. Anti-foam/defoamer formulations can be oil based. For example, oil based anti-foam compositions can be used to control foam in water based systems.

In some embodiments, an anti-foam composition according to the present disclosure includes a grain oil derived from a fermentation product. In some embodiments, the grain oil can include one or more (blends) of any of the grain oil compositions and byproducts of a grain oil composition as described herein. Nonlimiting examples of grain oil composition include a grain oil composition feedstock or a grain oil product (light phase) as described herein. Nonlimiting examples of a by-product of grain oil composition include an emulsion phase or a dehydrated emulsion phase (heavy phase) as described herein above. Blends of one or more of these grain oils can also be used. In some embodiments, the heavy phase can provide better foam reduction as shown in, e.g., Table 7 in the Examples section below.

However, the light phase may be desirable in cases where downstream processes include catalysts that can be poisoned by metals that may be present in the heavy phase.

In some embodiments, the light phase and/or heavy phase can also be used as a carrier oil component in defoamer compositions containing other active ingredients.

In some embodiments, one or more additional oils can be combined with a grain oil derived from a fermentation product to form a carrier oil component. Selection of a carrier oil can be for many different reasons including price, availability, biodegradability, and being a renewable product rather than a petroleum based product. A wide variety of plant and non-plant oils can be included in the carrier oil component. For example, the carrier oil component can also include mineral oil. The light phase and/or heavy phase described herein can also be used to replace at least a portion of mineral oil as a carrier oil component in defoamer compositions containing other active ingredients. For example, a grain oil derived from a fermentation product can be mixed with mineral oil in a wide range of amounts to form an anti-foam composition that can flow and spread throughout a composition so as to reduce or prevent the foaming of the composition. In some embodiments, the weight ratio of the grain oil derived from a fermentation product to the mineral oil can be in the range from to 1:90 to 1:1 or even less than 1:1, or even from 1:4 to less than 1:1.

A grain oil derived from a fermentation product can be combined with one or more anti-foaming ingredients/additives in a wide range of amounts depending on the final application. Nonlimiting examples of anti-foaming ingredients/additives include dimethylpolysiloxane, formaldehyde polyacrylic acid; mineral oil; polyethylene glycol (400) dioleate; [alpha]-hydro-omega-hydroxy-poly (oxyethylene)/poly(oxypropylene) (minimum 15 moles)/poly(oxyethylene); polyethylene glycol; polyoxyethylene 40 monostearate; polysorbate 60; polysorbate 65; propylene glycol alginate; silicon dioxide; sorbitan monostearate; aluminum stearate; butyl stearate; BHA; BHT; calcium stearate; fatty acids; formaldehyde; hydroxylated lecithin; isopropyl alcohol; magnesium stearate; petroleum wax; oleic acid; synthetic isoparaffinic petroleum hydrocarbons; oxystearin; polyoxyethylene dioleate; polyoxyethylene monoricinoleate; polypropylene glycol; polysorbate 80; potassium stearate; propylene glycol mono- and diesters of fats and fatty acids; soybean oil fatty acids(hydroxylated); tallow (hydrogenated, oxidized or sulfated), and mixtures thereof. In some embodiments, an anti-foam composition includes at least one hydrophobic particle component. A hydrophobic particle component can include hydrophobic wax particles, one or more hydrophobic silica particles, and mixtures thereof. In some embodiments, an anti-foam composition contains no detectable amount of phospholipid.

An anti-foam composition can be provided with a viscosity to facilitate one or more functions such as storage, transfer, application, and ability to prevent or reduce foam. A desirable viscosity or range of viscosities can depend on a variety of factors. Also, an anti-foam composition can be blended with one or more compositions to modify its viscosity. For example, if a grain oil derived from a fermentation product (e.g., dehydrated emulsion phase) is too thick, e.g., to pump, a grain oil derived from a fermentation product could be blended with mineral oil to reduce viscosity. Material handling issues, such as a thick vegetable oil anti-foam composition not flowing out of a tank or railcar could also be alleviated by adding mineral oil to reduce viscosity. Also, a lower viscosity anti-foam may disperse in a composition to be treated better than a higher viscosity antifoam. For example, an anti-foam with the viscosity of toothpaste may not disperse as readily as a lower-viscosity antifoam (when added to a process stream that needs to be defoamed or when added to the top of a foaming tank).

In some embodiments, an anti-foam composition and/or at least one of the grain oil composition or the byproduct of a grain oil composition has a Brookfield viscosity in the range from 10-300 centiPoise (cP) when measured at 22° C. and 100 rpm with a #31 spindle. In some embodiments, an anti-foam composition and/or at least one of the grain oil composition or the byproduct of a grain oil composition has a Brookfield viscosity in the range from 5-100 centiPoise (cP) when measured at 22° C. and 100 rpm with a #18 spindle. In some embodiments, an anti-foam composition and/or at least one of the grain oil composition or the byproduct of a grain oil composition has a Brookfield viscosity in the range from 5-100 centiPoise (cP) when measured at 22° C. and 50 rpm with a #18 spindle.

An anti-foam composition as described herein can be used to control foam in a foamable composition by mixing an amount of an anti-foam composition with the foamable composition to prevent, mitigate, or reduce foaming of the foamable composition. Foamable compositions that can benefit from an anti-foam composition include compositions that foam when subjected to agitation or mixing. Nonlimiting examples of foamable composition include one or more of aerobic digestion streams, wastewater treatment; manure pit foam; pulp and paper processing; coatings; paint; agrochemicals; food and beverage manufacturing; and one or more biorefinery compositions/process streams such as a stillage composition/stream.

One example of a stillage composition includes a stillage composition derived from processing “cellulosic biomass” such as corn stover. A stillage composition derived from corn stover includes fermenting a cellulosic mash to form a cellulosic beer; separating the cellulosic beer into a liquid stillage stream and a lignin cake stream. The liquid stillage stream can be mixed with an amount of an anti-foam composition as described herein to reduce foaming.

The amount of anti-foam composition to combine with a foamable composition can vary depending on the type of foamable composition, the process conditions, and the like. In some embodiments, the anhydrous heavy phase is added at a rate of 10-500 ppm, 20-300 ppm, 50-200 ppm, or approximately 100 ppm.

Mineral Oil Replacement

Embodiments of the present disclosure also include reducing the amount of mineral oil carrier in a wide variety of compositions by replacing at least a portion of the mineral oil carrier in the composition with an amount of a grain oil composition derived from a fermentation product.

The amount of mineral oil replaced can depend on a variety of factors such as cost and functionality (e.g., viscosity). In some embodiments, the weight ratio of mineral oil replaced to the amount of a grain oil composition derived from a fermentation product is in the range from 0.5:1 to 1.5:1.

Following are exemplary embodiments of the present disclosure:

1. A method of refining a grain oil composition feedstock to provide a grain oil product, wherein the method comprises:

combining the grain oil composition feedstock with water to form an oil-water mixture having water in an amount of 5-50% based on the total volume of the oil-water mixture (v/v), wherein the grain oil composition comprises an impurity component;

exposing the oil-water mixture to a temperature in the range from 0° C. to 50° C. for a time period at least until the oil-water mixture forms at least an oil phase and an emulsion phase; and recovering at least a portion of the oil phase from the emulsion phase to form the grain oil product, wherein an amount of the impurity component in the emulsion phase is greater than an amount of the impurity component in the grain oil product.

2. The method of embodiment 1, wherein the water is in an amount of 15-25% based on the total volume of the oil water mixture (v/v).

3. The method of any preceding embodiment, wherein the oil-water mixture is exposed to a temperature in the range from 20° C. to 30° C.

4. The method of any preceding embodiment, wherein the time period is from 5 minutes to 5 hours.

5. The method any preceding embodiment, wherein recovering at least a portion of the oil phase from the emulsion phase to form the grain oil product comprises passing the emulsion phase and the oil phase through at least one centrifuge to separate at least a portion of the oil phase from the emulsion phase to form the grain oil product.

6. The method of any preceding embodiment, wherein the emulsion phase comprises water, oil, and at least a portion of the impurity component.

7. The method of any preceding embodiment, wherein the impurity component comprises at least one impurity chosen from phospholipids, metals, free fatty acids, esters, soaps, gums, waxes, phosphatides, sterols, odiferous volatiles, colorants, and combinations thereof

8. The method of any preceding embodiment, wherein the impurity component comprises an element component, wherein the element component comprises at least one element chosen from calcium, phosphorus, potassium, sodium, magnesium and combinations thereof.

9. The method of embodiment 8, wherein the grain oil product comprises no more than 100 ppm of the element component, and wherein the emulsion phase comprises at least 100 ppm of the element component.

10. The method of embodiments 8 or 9, wherein at least a portion of the element component is present as soap.

11. The method of any preceding embodiment, wherein the grain oil composition feedstock comprises a triglyceride component present in an amount of at least 70 percent by weight of the grain oil composition feedstock.

12. The method of any preceding embodiment, wherein the grain oil composition feedstock and/or the grain oil product have a moisture content of 30 weight percent or less.

13. The method of any preceding embodiment, wherein the grain oil composition feedstock and/or grain oil product contain no detectable phospholipid.

14. The method of any preceding embodiment, wherein the grain oil composition feedstock is derived from a grain chosen from corn, barley, rice, wheat, soybean, rapeseed, rye, and combinations thereof

15. The method of any preceding embodiment, wherein the grain oil composition feedstock is derived from a fermentation product, wherein the fermentation product is a stillage composition, wherein the stillage composition is derived from a grain material, wherein the grain material is chosen from corn, barley, rice, wheat, soybean, rapeseed, rye, and combinations thereof, and wherein the stillage composition is chosen from whole stillage, thin stillage, wet cake, syrup, and combinations thereof.

16. The method of embodiment 15, wherein the fermentation product is derived from a method comprising fermenting a grain mash composition to form a beer comprising a biochemical, wherein the grain mash composition comprises grain solids, grain oil and sugar, wherein fermenting comprises fermenting the grain mash in the presence of one or more enzymes to generate one or more fatty acid alkyl esters.

17. The method of embodiment 16 wherein the one or more enzymes are chosen from lipase, esterase, and combinations thereof, wherein the one or more enzymes are endogenous enzymes and/or exogenous enzymes, and wherein the one or more fatty acid alkyl esters comprise one or more fatty acid ethyl esters.

18. The method of embodiments 15, 16, or 17, wherein grain oil composition feedstock is derived from whole stillage by a method comprising:

separating whole stillage into thin stillage and wet cake;

optionally evaporating at least a portion of water from the thin stillage to condense the thin stillage into a syrup;

separating the thin stillage or syrup into a first oil fraction and a first aqueous fraction; and

adjusting pH of the first oil fraction to separate the first oil fraction into a second oil fraction and a second aqueous fraction, wherein the second oil fraction is the grain oil composition feedstock.

19. The method of embodiment 18, wherein the grain oil composition feedstock is at a temperature greater than 70° C., and further comprising cooling the grain oil composition feedstock to a temperature in the range from 0° C. to 50° C.

20. The method of any preceding embodiment, wherein the oil phase has a first bulk density and the emulsion phase has a second bulk density, wherein the first bulk density is less than the second bulk density.

21. The method of any preceding embodiment, further comprising dehydrating the emulsion phase to produce a dehydrated emulsion product, wherein the dehydrated emulsion product comprises:

a moisture content of 1% or less based on the total weight of the dehydrated emulsion product;

a triglyceride component; and

at least 100 ppm of an element component, wherein the element component comprises at least one element chosen from calcium, phosphorus, potassium, sodium, and combinations thereof.

22. The method of embodiment 21, further comprising mixing at least one anti-foaming ingredient with the grain oil composition feedstock, the grain oil product, the emulsion phase, the dehydrated emulsion product, or blends thereof to form an anti-foam composition.

23. The method of any preceding embodiment, further comprising filtering the grain oil product to remove at least a portion of a particle component.

24. A system for refining a grain oil composition feedstock comprising:

a) a source of a grain oil composition feedstock, wherein the grain oil composition comprises an impurity component;

b) a source of water;

c) a first system in fluid communication with the source of the grain oil composition and the source of the water, wherein the first system is configured to:

i) combine and mix the grain oil composition and the water to form an oil-water mixture having water in an amount of 5-50% based on the total volume of the oil water mixture (v/v); and

ii) expose the oil-water mixture to a temperature in the range from 0° C. to 50° C. for a time period at least until the oil-water mixture forms at least an oil phase and an emulsion phase; and

d) a separation system configured to recover at least a portion of the oil phase from the emulsion phase to form the grain oil product.

25. The system of embodiment 24, wherein the first system comprises one or more static mixers, impeller mixers, pumps, shear mixers, tank recirculation loops, tank mixers, and combinations thereof

26. The system of embodiments 24 or 25, wherein the separation system comprises one or more centrifuges, decanter centrifuges, disk stack centrifuges, screen centrifuges, hydrocyclones, and combinations thereof.

27. The system of any of embodiments 24-26, further comprising:

a) a distillation system configured to provide a source of whole stillage stream;

b) a first separation system in fluid communication with the distillation system and configured to separate the whole stillage stream into a thin stillage stream and a wet cake stream;

c) an evaporation system in fluid communication with the first separation system and configured to condense the thin stillage stream into a syrup stream;

d) a second separation system in fluid communication with the evaporation system and configured to separate the syrup stream into a first aqueous phase stream and a first oil phase stream; and

e) a third separation system in fluid communication with the second separation system and configured to separate the first oil phase stream into a second aqueous phase stream and a second oil phase stream, wherein the second oil phase stream is the source of the grain oil composition feedstock,

wherein the separation system configured to recover at least a portion of the oil phase from the emulsion phase is a fourth separation system.

28. The system of any of embodiments 24-27, wherein at least one of the first separation system, second separation system, and third separation system comprises one or more centrifuges, decanter centrifuges, disk stack centrifuges, screen centrifuges, hydrocyclones, and combinations thereof

An anti-foam composition comprising:

a) a carrier oil component comprising a grain oil derived from a fermentation product, wherein the grain oil is chosen from a grain oil composition, a byproduct of a grain oil composition, and mixtures thereof; and

b) at least one anti-foaming ingredient.

30. The anti-foam composition of embodiment 29, wherein at least one of the grain oil composition or the byproduct of a grain oil composition comprises a fatty acid alkyl ester component, wherein the fatty acid alkyl ester component comprises one or more fatty acid alky esters.

31. The anti-foam composition of embodiments 29 or 30, wherein the fatty acid alkyl ester component is present in an amount of 0.1 percent or more by weight based on the total weight of the anti-foam composition.

32. The anti-foam composition of embodiment 29, 30, or 31, wherein at least one of the grain oil composition or the byproduct of a grain oil composition further comprises a free fatty acid component comprising one or more free fatty acids, wherein the free fatty acid component is present in an amount of at least 0.1 percent or more by weight based on the total weight of the anti-foam composition.

33. The anti-foam composition of embodiment 32, wherein the free fatty acid component and the fatty acid alkyl ester component are generated during a grain ethanol production process, wherein the free fatty acid component and/or fatty acid alkyl ester component are endogenous and/or chemically formed in-situ.

34. The anti-foam composition of any of embodiments 29-33, wherein at least one of the grain oil composition or the byproduct of a grain oil composition is derived from distiller's corn oil.

35. The anti-foam composition of any of embodiments 29-34, wherein the anti-foam composition contains no detectable amount of phospholipid.

36. The anti-foam composition of any of embodiments 29-34, wherein the anti-foaming ingredient comprises a hydrophobic particle component.

37. The anti-foam composition of embodiment 36, wherein the hydrophobic particle component is chosen from one or more hydrophobic wax particles, one or more hydrophobic silica particles, and mixtures thereof.

38. The anti-foam composition of any of embodiments 29-37, wherein at least one of the grain oil composition or the byproduct of a grain oil composition further comprise an element component, wherein the element component comprises at least one element chosen from calcium, phosphorus, potassium, sodium, and combinations thereof

39. The anti-foam composition of embodiment 38, wherein at least a portion of the element component is present as soap.

40. The anti-foam composition of any of embodiments 29-39, wherein the anti-foam composition and/or at least one of the grain oil composition or the byproduct of a grain oil composition has a Brookfield viscosity in the range from 10-300 centiPoise (cP) when measured at 22° C. and 100 rpm with a #31 spindle.

41. The anti-foam composition of any of embodiments 29-40, wherein the anti-foam composition and/or at least one of the grain oil composition or the byproduct of a grain oil composition has a Brookfield viscosity in the range from 5-100 centiPoise (cP) when measured at 22° C. and 100 rpm with a #18 spindle.

42. The anti-foam composition of any of embodiments 29-41, wherein the anti-foam composition and/or at least one of the grain oil composition or the byproduct of a grain oil composition has a Brookfield viscosity in the range from 5-100 centiPoise (cP) when measured at 22° C. and 50 rpm with a #18 spindle.

43. The anti-foam composition of any of embodiments 29-42, wherein the carrier oil component further comprises mineral oil.

44. The anti-foam composition of embodiment 43, wherein the weight ratio of the grain oil composition and/or the byproduct of a grain oil composition to the mineral oil is in the range from to 1:90 to 1:1.

45. The anti-foam composition of any of embodiments 29-44, wherein the carrier oil component comprises the grain oil derived from a fermentation product in an amount from 50 to 100 percent by weight of the total carrier oil component.

46. A method of controlling foam in a foamable composition, wherein the method comprises mixing a grain oil with the foamable composition to prevent, mitigate, or reduce foaming of the foamable composition, wherein the grain oil is derived from a fermentation product, and wherein the grain oil is chosen from a grain oil composition, a byproduct of a grain oil composition, and mixtures thereof.

47. The method of embodiment 46, wherein at least one of the grain oil composition and the byproduct of a grain oil composition has a fatty acid alkyl ester component.

48. The method of embodiments 46 or 47, wherein the foamable composition is a stillage 5 composition.

49. The method of embodiment 46, 47, or 48, wherein the stillage composition is a liquid stillage stream provided by a process comprising:

a) fermenting a cellulosic mash to form a cellulosic beer;

b) separating the cellulosic beer into a liquid stillage stream and a lignin cake stream;

c) mixing the liquid stillage stream with an amount of at least one of the grain oil composition and the byproduct of a grain oil composition to reduce foaming.

50. The method of embodiment 49, wherein the cellulosic mash is derived from ground corn stover.

51. The method of any of embodiments 46-50, wherein the fatty acid alkyl ester component comprises one or more fatty acid alkyl esters, and wherein the fatty acid alkyl ester component is present in an amount of 10 percent or more by weight based on the total weight of the grain oil composition or the byproduct of a grain oil composition.

52. A method of reducing the amount of mineral oil carrier in a composition, wherein the method comprises replacing at least a portion of the mineral oil carrier in the composition with an amount of a grain oil composition derived from a fermentation product.

53. The method of embodiment 52, wherein the weight ratio of mineral oil replaced to the amount of a grain oil composition derived from a fermentation product is in the range from 0.5:1 to 1.5:1.

54. The method of embodiment 52 or 53, wherein all of the mineral oil is replaced with the grain oil composition derived from a fermentation product.

### EXAMPLES

The following examples are intended to illustrate different aspects and embodiments of the present disclosure. It will be recognized that various modifications and changes may be made from the experimental embodiments described herein without departing from the scope of the claims. In these examples, as well as elsewhere in the application, all ratios, parts, and percentages are by weight unless otherwise indicated or apparent from the context.

### Example 1

Distiller's corn oil from five different sources was obtained and tested as described herein. To each 50 ml centrifuge tube were added 40 ml of room temperature distiller's corn oil and 10 ml of water (RO) to make a 20 vol % mixture of water and oil. The mixture was vigorously mixed using a vortex mixer to form an emulsion. Each of the tubes were centrifuged on a bench top centrifuge that had been chilled to 4° C. and spun at 4500 rpm for 30 minutes. Each sample was removed from the centrifuge and using vacuum suction the supernatant from the top was removed from each tube. The process resulted in a clarified, light phase (top layer) (corn oil product) and a heavy phase (bottom layer) (emulsion phase). Table 3 shows the percentage of the clarified, light phase and heavy phase.

TABLE 3

| Sample ID | % Clarified Phase | % Heavy Phase |
|---|---|---|
| DCO 1 | 96.57 | 3.43 |
| DCO 2 | 89.90 | 10.10 |
| DCO 3 | 92.86 | 7.14 |
| DCO 4 | 79.25 | 20.75 |
| DCO 5 | 93.27 | 6.73 |
| Mean | 90.37 | 9.63 |

Table 4 shows the concentration of metals in ppm for each of the sample oils before the treatment with water and separation.

TABLE 4

| Sample ID | Calcium | Magnesium | Phosphorus | Potassium | Sodium |
|---|---|---|---|---|---|
| DCO 1 | 13 | 103 | 49 | 40 | 81 |
| DCO 2 | 10 | 64 | 41 | 30 | 60 |
| DCO 3 | 1 | 10 | 6 | 44 | 72 |
| DCO 4 | 45 | 219 | 142 | 37 | 111 |
| DCO 5 | 1 | 10 | 7 | 25 | 53 |

Table 5 show the concentration of metals in ppm for each of the sample oils after the treatment with water and separation as measured in the clarified, light phase.

TABLE 5

| Sample ID | Calcium | Magnesium | Phosphorus | Potassium | Sodium |
|---|---|---|---|---|---|
| DCO 1 Clarified | <1 | 1 | 3 | 2 | 3 |
| DCO 2 Clarified | <1 | 2 | 3 | <2 | 4 |
| DCO 3 Clarified | <1 | 1 | 3 | 5 | 7 |
| DCO 4 Clarified | 1 | 7 | 5 | 4 | 9 |
| DCO 5 Clarified | <1 | 1 | 3 | 5 | 5 |

The metals were determined according to AOCS Ca 17-01, were xylene was used as the solvent.

### Example 2

Distiller's corn oil obtained from a fermentation plant passed through a heat exchanger to cool/heat the oil to ˜65° F. Water was introduced into a pipe at a rate to make a 15 vol % water solution. The oil/water mix was passed through an inline pipe mixer to form an emulsion. The mixed oil/water mixture was then processed through a nozzle clarifier centrifuge resulting in a clarified light phase (corn oil product) and a heavy phase (emulsion phase). The light and heavy phase was split 79% clarified phase and 21% heavy phase.

Table 6 shows the metals for untreated distiller corn oil (DCO) compared to distillers corn oil processed (DCO clarified).

TABLE 6

| Sample ID | Calcium | Magnesium | Phosphorus | Potassium | Sodium |
|---|---|---|---|---|---|
| DCO | 18 | 125.5 | 82.5 | 46.5 | 98.5 |
| DCO Clarified | 2.9 | 5.8 | 3.1 | 3.3 | 2.5 |

It can be seen that the addition of water in the separation process aids in reducing the metal content in the clarified oil phase.

### Example 3

Samples of a heavy phase (emulsion phase as obtained from the preparation of the oil/water mixture and then separated as described in Example 1) were dried by evaporating the water on a hot plate. The oil was Voila® corn oil. The dried emulsion was then bleached using heat and the addition of hydrogen peroxide to produce a material with a lighter color that when re-emulsified with water appeared white. The dried emulsion was extracted with acetone yielding an oil fraction and a de-oiled precipitate fraction.

### Example 4

Samples of the heavy phase (emulsion phase as obtained from the preparation of the oil/water mixture and then separated) were dried via evaporation to form an anhydrous heavy phase. The anhydrous heavy phase was used in place of a commercial anti-foam product. The anhydrous heavy phase was trickled into a wastewater treatment process at a range of 50-200 ppm to reduce foaming during wastewater treatment. Foaming was controlled to a level comparable to commercial anti-foam products typically used in wastewater treatment facilities.

### Example 5

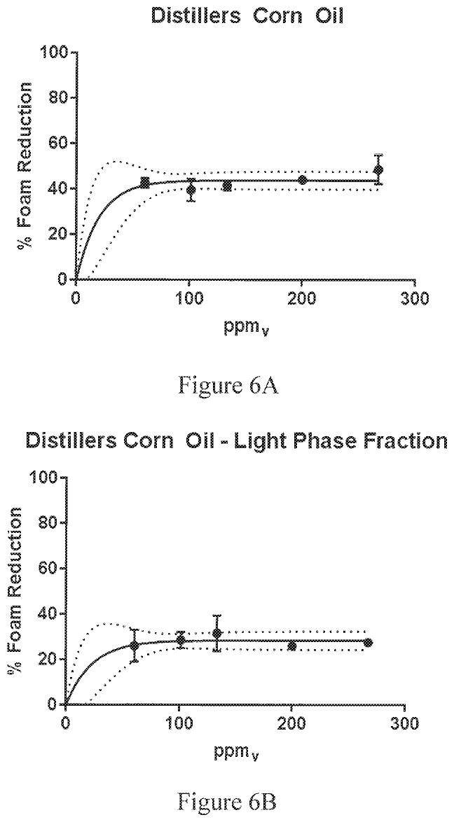

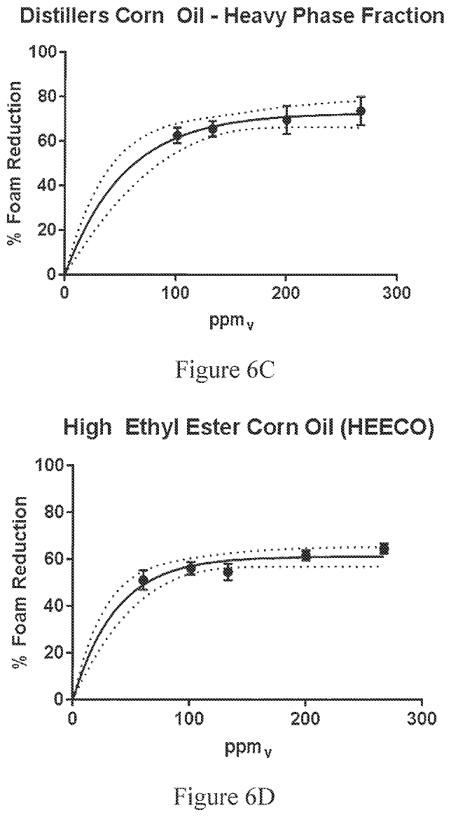

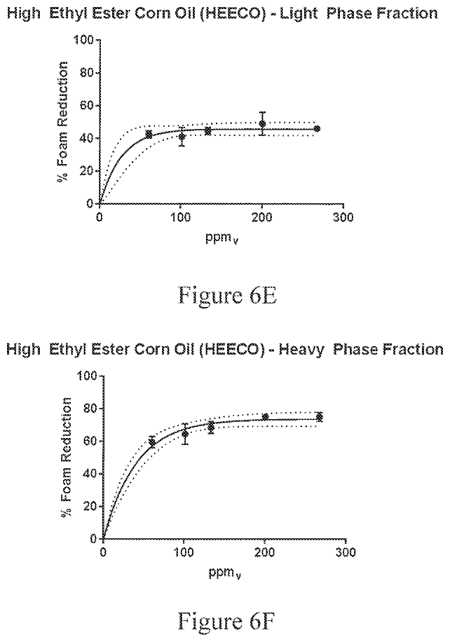

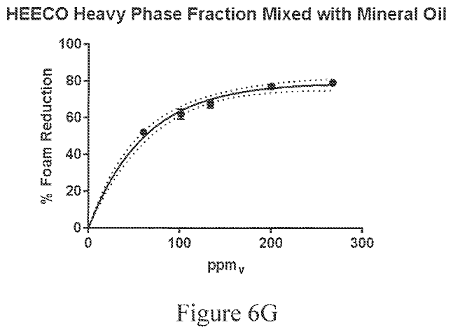

FIGS. 6A through 6G and Table 7 show percent foam reduction when adding different antifoams (defoamers) at varying concentrations to an aqueous solution of sodium lauryl ether sulfate (SLES). The aqueous solution contained 0.1 mass % SLES in water. After performing an antifoam testing procedure, the volume of foam with and without antifoam was used to calculate a volume-percent foam reduction. Foam reduction data was generated based on the following modified version ASTM test method: E2407-05(2015) Standard Test Method for Effectivenes of Defoaming Agents. The antifoam testing procedure used to measure the effectiveness of various antifoams is shown below.

Antifoam Testing Procedure:

1. Load 250 ml of substrate (0.1 mass % sodium lauryl ether sulfate (SLES) in water or other substrate) into a Wearing blender that contains a glass vessel.

2. Reduce the power to the blender to 60% of full power.

3. Blend the substrate for 30 seconds.

4. Let the blended substrate stand 3 minutes.

5. Record the liquid level in a spreadsheet.

6. Record the foam level in the spreadsheet.

7. Add the correct volume of antifoam agent to the substrate using a repeater pipette.

8. Blend the substrate and antifoam agent mixture for 30 seconds.

9. Let the substrate and antifoam agent mixture stand for 6 minutes

10. Record the liquid level in the spreadsheet.

11. Record the foam level in the spreadsheet.

12. Calculate the foam reduction using the volume of foam that remained when antifoam agent was not present (after step 4—initial foam volume) and the volume of foam that remained when antifoam agent was present (after step 9—final foam volume)*.

* Percent foam reduction was calculated by subtracting the final foam volume from the initial foam volume. This difference in volume was then divided by the initial foam volume and multiplied by 100 to obtain a volume percent foam reduction.

13. Rinse the vessel with hot water.

FIGS. 6A through 6G show volume percent foam reduction for seven different corn oil based antifoams at varying dose rates when using a model substrate (0.1 mass % SLES in water). The dose rates are reported as parts per million by volume (ppmv).

FIG. 6A shows foam reduction data when using DCO having a fatty acid ethyl esters content of about 10 percent by weight based on the total weight of the DCO as antifoam.

FIG. 6B shows foam reduction data when using DCO light phase fraction as anti-foam. DCO light phase fraction refers to the light phase fraction of DCO obtained from the DCO in FIG. 6A and isolated by centrifuging DCO and removing the top, clarified layer of corn oil. Alternatively, the fractions can be isolated by gravity settling DCO and removing the top, clarified layer of corn oil.

FIG. 6C shows foam reduction data when using DCO heavy phase fraction as anti-foam. DCO heavy phase fraction refers to the heavy, bottom layer of DCO obtained from the DCO in FIG. 6A.

FIG. 6D shows foam reduction data when using high ethyl ester corn oil (HEECO) as anti-foam. The HEECO sample used to generate the data shown in FIG. 6D through FIG. 6G contained approximately 60% fatty acid ethyl esters. As mentioned above, HEECO can be produced by adding a lipase or esterase during the fermentation step at a biorefinery. When a lipase or esterase is added to fermentation, the corn oil that is subsequently separated contains a higher concentration of fatty acid ethyl esters and may have enhanced utility to act as an antifoam. In this example, a lipase addition of approximately 25 ppm in fermentation resulted in a corn oil that contained approximately 60% ethyl esters. Alternatively, in a process separate from fermentation, DCO can be combined with ethanol in the presence of a catalyst such as an acid, base, or lipase to generate HEECO. Comparing FIG. 6D to FIG. 6A illustrates that corn oil exposed to a lipase or esterase has enhanced antifoam capability, compared to corn oil not exposed to a lipase or esterase.

FIG. 6E shows foam reduction data when using HEECO light phase fraction as anti-foam. HEECO light phase fraction refers to the light phase fraction of a high ethyl ester corn oil and in this example was the light phase separated from the HEECO of FIG. 6D.

FIG. 6F shows foam reduction data when using HEECO heavy phase fraction as anti-foam. HEECO heavy phase fraction refers to the heavy phase fraction of a high ethyl ester corn oil and in this example was the heavy phase separated from the HEECO of FIG. 6D. These heavy and light phases can be isolated in a similar fashion as described above for DCO light phase fraction and DCO heavy phase fraction.

FIG. 6G shows foam reduction data for a sample that was prepared by combining HEECO heavy phase fraction with mineral oil in a ratio to form a mixture comprising approximately 25 mass % HEECO heavy phase fraction in mineral oil. In some examples, a lower concentration of HEECO heavy phase in mineral oil may be desirable, e.g., to lower the viscosity of the mixture and make the antifoam composition more flowable. Flowability may be important for applications where antifoam is pumped, transported through pipes, or removed from storage containers or vessels. In such examples, the concentration of HEECO heavy phase in mineral oil may range, e.g., from 0.01 to 25 mass %, including the end points of this range. In other examples, higher concentration of HEECO heavy phase fraction in mineral oil may be desirable, e.g., to reduce shipping costs of the HEECO heavy phase fraction active ingredient. In such examples, the concentration of HEECO heavy phase in mineral oil may range from 25 to 50 mass %, including the end points of this range.

Table 7 is a table that shows the volume percent foam reduction for DCO, DCO light phase fraction, DCO heavy phase fraction, HEECO, HEECO light phase fraction, HEECO heavy phase fraction, and a mixture of HEECO heavy phase fraction and mineral oil when using a model substrate (0.1 mass % SLES in water). The values in Table 7 are the point at which the curves in FIGS. 6A through 6G plateau. As shown in Table 7, the DCO heavy phase fraction resulted in a greater volumetric foam reduction, compared to the DCO sample. Similarly, the HEECO heavy phase fraction resulted in a greater volumetric foam reduction, compared to the HEECO sample. This result may be advantageous in that the DCO light phase fraction and the HEECO light phase fraction are clear with a homogeneous appearance. These oil characteristics may be desirable for certain vegetable oil customers. A corn oil separation process that produces a heavy phase fraction, that can be used as an anti-foam, and a light phase fraction, which is clear and homogeneous, may be beneficial because it converts a commodity corn oil into two potentially higher value products.

TABLE 7

| | | | | | | | HEECO |
|---|---|---|---|---|---|---|---|
| | | | | | | | Heavy |
| | | | | High Ethyl | | HEECO | Phase |
| | | DCO Light | DCO Heavy | Ester Corn | HEECO | Heavy | Fraction |
| | Distillers | Phase | Phase | Oil | Light Phase | Phase | Mixed with |
| | Corn Oil | Fraction | Fraction | (HEECO) | Fraction | Fraction | Mineral Oil |
| Volume % | 43.7 | 28.38 | 72.6 | 61.07 | 45.81 | 73.7 | 79.04 |
| Foam | | | | | | | |
| Reduction | | | | | | | |
| (Plateau) | | | | | | | |
| Density | 0.916 | 0.916 | 0.934 | 0.890 | 0.883 | 0.928 | na |

Tables 8-10 show compositional analysis for the samples shown in FIGS. 6A-6F.

Table 8 shows elemental analysis data for the following samples: DCO, DCO light phase fraction from the DCO, DCO heavy phase fraction from the DCO, HEECO, HEECO light phase fraction from the HEECO, and HEECO heavy phase fraction from the HEECO. The HEECO sample in Table 8 contained approximately 60% ethyl esters.

Table 9 shows fatty acid composition data for the following samples: DCO, DCO light phase fraction from the DCO, DCO heavy phase fraction from the DCO, HEECO, HEECO lightphase fraction from the HEECO, and HEECO heavy phase fraction from the HEECO. The HEECO sample in Table 9 contained approximately 60% ethyl esters.

Table 10 shows mono- and diglyceride concentration and soap concentration data for the following sample: DCO, DCO light phase fraction from the DCO, DCO heavy phase fraction from the DCO, HEECO, HEECO light phase fraction from the HEECO, and HEECO heavy phase fraction from the HEECO. Table 8 also shows wax concentration data for a sample of DCO heavy phase fraction and a sample of HEECO heavy phase fraction. The HEECO sample in Table 10 contained approximately 60% ethyl esters. The compositional differences between the seven samples shown in Tables 8-10 may be responsible for the varying effectiveness in foam reduction when using these samples in antifoam applications.

TABLE 8

| | | | | High | | | |
|---|---|---|---|---|---|---|---|
| | | DCO | DCO | Ethyl | HEECO | HEECO | |
| | Distillers | Light | Heavy | Ester | Light | Heavy | |
| Elemental | Corn Oil | Phase | Phase | Corn Oil | Phase | Phase | |
| Analysis | (DCO) | Fraction | Fraction | (HEECO) | Fraction | Fraction | Units |
| Calcium | 38.50 | 0.98 | 25.30 | 62.50 | 37.00 | 171.00 | ppm |
| Magnesium | 232.00 | 1.76 | 83.50 | 207.00 | 32.10 | 1,810.00 | ppm |
| Phosphorus | 162.00 | 0.99 | 56.60 | 59.30 | 9.89 | 569.00 | ppm |
| Potassium | 60.10 | 15.90 | 112.00 | 103.00 | 89.40 | 293.00 | ppm |
| Sodium | 130.00 | 18.60 | 382.00 | 578.00 | 547.00 | 1,3700 | ppm |

TABLE 9

| | | | | High | | | |
|---|---|---|---|---|---|---|---|
| | | DCO | DCO | Ethyl | HEECO | HEECO | |
| | Distillers | Light | Heavy | Ester | Light | Heavy | |
| Fatty Acid | Corn Oil | Phase | Phase | Corn Oil | Phase | Phase | |
| Composition | (DCO) | Fraction | Fraction | (HEECO) | Fraction | Fraction | Units |
| C8 Caprylic | | | | | | 0.50 | Mg FA/g |
| C10 Capric | | | | | | 0.40 | Mg FA/g |
| C14 Myristic | 0.50 | 0.40 | 0.40 | 0.60 | 0.60 | 0.50 | Mg FA/g |
| C16 Palmitic | 124.90 | 119.90 | 146.10 | 123.10 | 118.60 | 168.70 | Mg FA/g |
| C16:1n7 Palmitoleic | 1.70 | 1.70 | 1.50 | 1.60 | 1.60 | 1.20 | Mg FA/g |
| C17 Margaric | 0.60 | 0.70 | 0.80 | 0.60 | 0.60 | 0.90 | Mg FA/g |
| C18 Stearic | 18.00 | 17.60 | 22.00 | 18.10 | 17.20 | 26.40 | Mg FA/g |
| C18:1n9 Oleic | 248.10 | 253.60 | 231.40 | 234.10 | 241.00 | 192.40 | Mg FA/g |
| C18:1n7 Vaccenic | 4.90 | 5.40 | 4.40 | 5.50 | 4.70 | 4.30 | Mg FA/g |
| C18:2 Linoleic | 484.30 | 492.90 | 446.80 | 448.10 | 460.60 | 363.80 | Mg FA/g |
| C18:3n3 alpha- Linolenic | 11.90 | 12.40 | 11.20 | 11.70 | 12.00 | 9.40 | Mg FA/g |
| C20 Arachidic | 3.80 | 3.60 | 4.50 | 3.70 | 3.40 | 6.20 | Mg FA/g |
| C20:1 Eicosenoic | 3.20 | 3.30 | 2.90 | 3.20 | 3.30 | 2.70 | Mg FA/g |
| C20:2n6 Eicosadienoic | 0.30 | 0.30 | 0.20 | 0.30 | 0.40 | 0.30 | Mg FA/g |
| C22 Behinic | 1.60 | 1.30 | 2.90 | 1.60 | 1.30 | 4.00 | Mg FA/g |
| C24 Lignoceric | 2.00 | 1.70 | 3.40 | 2.10 | 1.80 | 5.00 | Mg FA/g |
| Others | 1.60 | 2.40 | 1.80 | 2.60 | 3.00 | 2.50 | Mg FA/g |
| Total Fatty Acid | 907.40 | 917.20 | 880.30 | 856.90 | 870.10 | 789.20 | Mg FA/g |
| Total Saturates | 151.40 | 145.20 | 180.10 | 149.80 | 143.50 | 212.60 | Mg FA/g |
| Total Monounsaturates | 257.90 | 264.00 | 240.20 | 244.40 | 250.60 | 200.60 | Mg FA/g |
| Total Polyunsaturates | 496.50 | 505.60 | 458.20 | 460.10 | 473.00 | 373.50 | Mg FA/g |
| Total Omega 3 | 11.90 | 12.40 | 11.20 | 11.70 | 12.00 | 9.40 | Mg FA/g |
| Total Omega 6 | 484.60 | 493.20 | 447.00 | 448.40 | 461.00 | 364.10 | Mg FA/g |
| Total Omega 9 | 251.30 | 256.90 | 234.30 | 237.30 | 277.30 | 195.10 | Mg FA/g |
| Free Fatty Acids as | 4.51 | 6.82 | 11.90 | 7.01 | 8.42 | 14.30 | % |
| Oleic | | | | | | | |

TABLE 10

| | | | | High | | | |
|---|---|---|---|---|---|---|---|
| | | DCO | DCO | Ethyl | HEECO | HEECO | |
| Mono & | Distillers | Light | Heavy | Ester | Light | Heavy | |
| Diglycerides | Corn Oil | Phase | Phase | Corn Oil | Phase | Phase | |
| by HPLC | (DCO) | Fraction | Fraction | (HEECO) | Fraction | Fraction | Units |
| Diglycerides | 3.23 | 2.96 | 3.15 | 5.29 | 5.35 | 4.30 | % (w/w) |
| Monoglycerides | 0.06 | 0.07 | 0.19 | 0.36 | 0.37 | 0.39 | % (w/w) |
| Soaps | 1,170.00 | 311.00 | 8,880.00 | 20,700.00 | 877.00 | 213,000.00 | ppm |
| Total Waxes | | | 893 | | | 215 | mg/Kg |

### Example 6

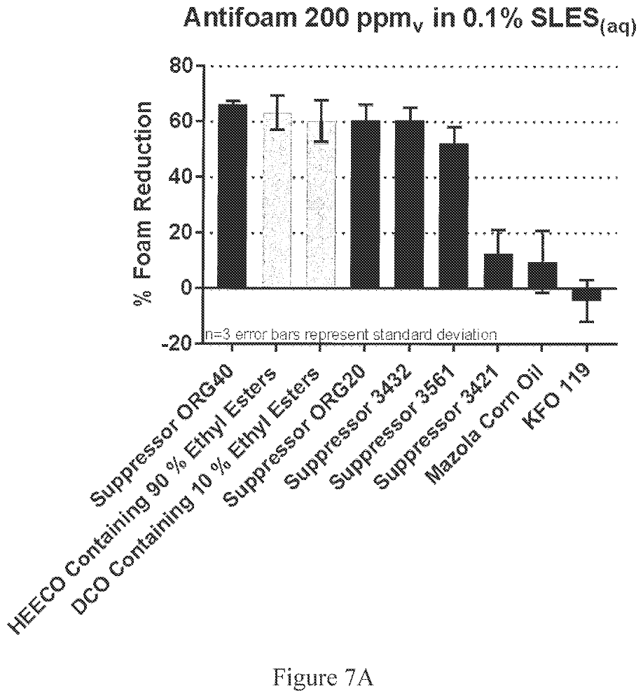

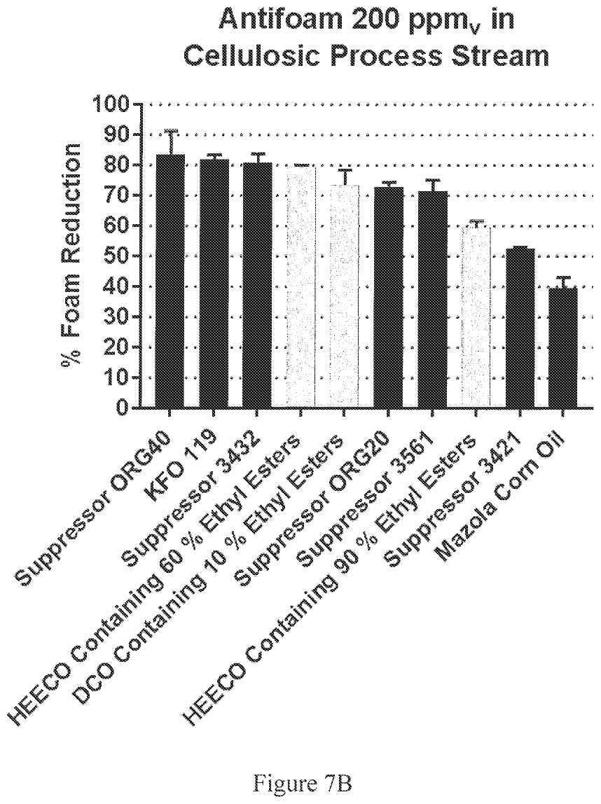

FIG. 7A shows the volume percent foam reduction for a HEECO sample containing 90% ethyl esters, a DCO sample containing 10% ethyl esters, six commercially available antifoam products, and food-grade, refined Mazola corn oil. The HEECO containing 90% ethyl esters described herein was produced by combining DCO with ethanol in the presence of lipase. The data in FIG. 7A was generated by following the antifoam testing procedure, outlined above. An aqueous solution of sodium lauryl ether sulfate (SLES) was used as substrate. The aqueous solution of SLES contained 0.1 mass % SLES in water. Antifoam was added at a dose rate of 200 ppmv.

FIG. 7B shows the volume percent foam reduction for a HEECO sample containing 90% ethyl esters, a HEECO sample containing 60% ethyl esters, a DCO sample containing 10% ethyl esters, six commercially available antifoam products, and food-grade, refined Mazola corn oil. The data in FIG. 7B was generated by following the antifoam testing procedure, outlined above except using an evaporated thin stillage sample from a cellulosic ethanol facility as substrate. Antifoam was added at a dose rate of 200 ppmv when generating the data shown in FIG. 7B.

### Example 7

Example 7 measured Brookfield viscosity of various corn oil samples using a Brookfield viscometer having model number DV2TLVTJ0 at the conditions identified in Table 11 below and using a small sample adapter to maintain the temperature of the sample at 22C using a water bath.

TABLE 11

| | | 50% Heeco | 50% Heeco | | | | | |
|---|---|---|---|---|---|---|---|---|
| | | Heavy + | Heavy + | | | | | |
| | | 50% | 75% | | | | | |
| | Heeco | Mineral | Mineral | | Heeco | Heeco | | DCO |
| | Heavy | Oil | Oil | Heeco | Light | Heavy | DCO | Light |
| Viscosity | 165.9- | 37.8- | 24.96- | 41.94- | 17.10- | 224.7- | 60.0- | 39.3- |
| (centiPoise) | 169.2 | 38.4 | 25.11 | 42.84 | 17.16 | 232.2 | 63.0 | 39.9 |
| RPM | 100 | 100 | 100 | 50 | 100 | 100 | 100 | 100 |
| Spindle | 31 | 31 | 18 | 18 | 18 | 31 | 31 | 31 |
| Temperature ° C. | 22 | 22 | 22 | 22 | 22 | 22 | 22 | 22 |

### Example 8

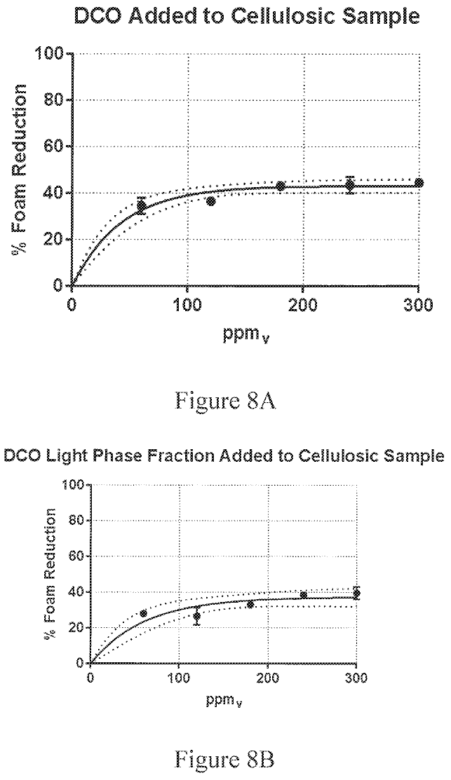

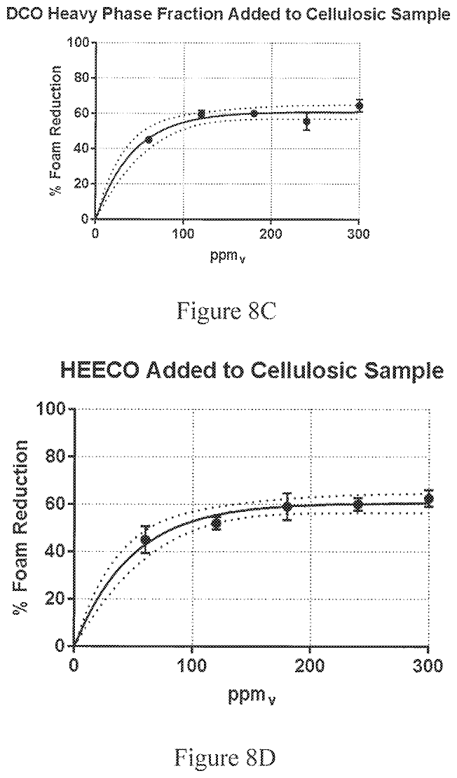

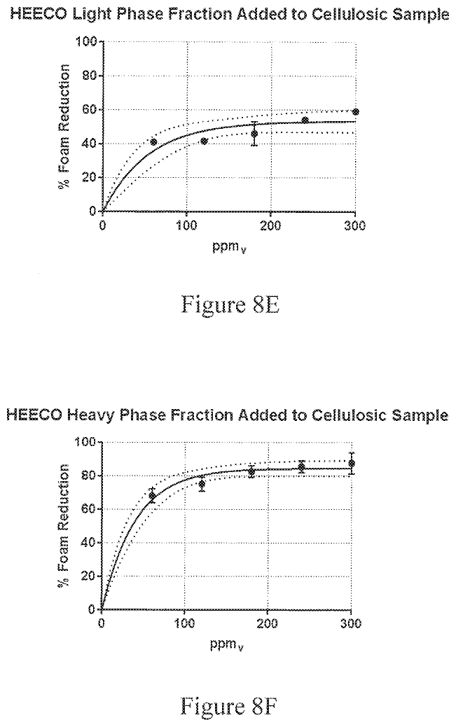

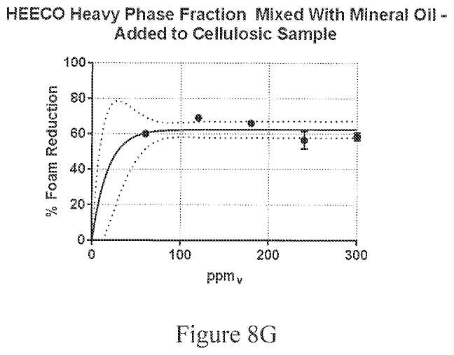

The sample of DCO in FIGS. 8A-C is the same sample as in FIG. 7B but was performed on a different day using a different foaming cellulosic substrate. The sample of HEECO used in FIGS. 8D-8G is a different sample than that used in FIG. 7B. DCO, DCO light phase fraction, DCO heavy phase fraction, HEECO, HEECO light phase fraction, HEECO heavy phase fraction, and HEECO heavy phase fraction mixed with mineral oil may be effective in reducing foam at cellulosic ethanol biorefineries, e.g. reducing foaming when concentrating liquids after solid liquid separation of cellulosic stillage. For example, the data in FIGS. 8A through 8G show that these corn oil based antifoams are effective at reducing foam in a sample of evaporated thin stillage from a cellulosic ethanol facility. The HEECO used to generate the data in FIGS. 8D through 8G contained approximately 60% ethyl esters. The antifoam testing procedure, outlined above, was used to generate the data shown in FIGS. 8A through 8G and an evaporated thin stillage sample from a cellulosic ethanol facility was used as substrate.

Table 12 is a table that shows the volume percent foam reduction. The values in Table 10 are the point at which the curves in FIGS. 8A through 8G plateau.

TABLE 12

| | | | | High | | | |
|---|---|---|---|---|---|---|---|
| | | DCO | DCO | Ethyl | HEECO | HEECO | HEECO |
| | Distillers | Light | Heavy | Ester | Light | Heavy | Heavy |
| | Corn Oil | Phase | Phase | Corn Oil | Phase | Phase | Phase/Mineral |
| | (DCO) | Fraction | Fraction | (HEECO) | Fraction | Fraction | Oil Mix |
| Volume % | 43.1 | 37.34 | 60.9 | 60.58 | 53.33 | 84.54 | 62.41 |
| Foam | | | | | | | |
| Reduction | | | | | | | |
| (Plateau) | | | | | | | |

### Example 9

An experiment was conducted with oils from different sources as a carrier oil in an anti-foam composition. Each anti-foam composition was prepared by adding an amount of “anti-foam” additive to the carrier oil. For each carrier oil from a given source, the amount of anti-foam additive was varied from 0 weight percent, 6 weight percent, and 12 weight percent based on the total weight of anti-foam composition. The anti-foam additive used in these tests was a 50:50 mixture of Dumacil® 100 FGK hydrophobic silica powder and Dumacil® 300 FGK hydrophobic silica powder, both of which are commercially available. Each resulting anti-foam composition was tested and analyzed for its anti-foam characteristics using a Ross Miles Foam Analyzer (RMFA) in accordance with ASTM D 1173. The test used a 0.1% solution of sodium lauryl ether sulfate (SLES) to generate a foam. Each anti-foam composition was dosed at 200 ppm. All results are n=4 with error bars representing standard deviation.

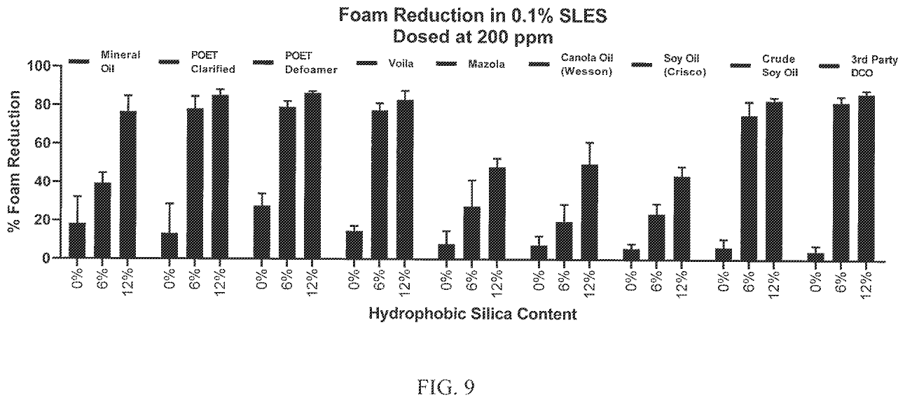

In FIG. 9, “POET clarified” and “POET defoamer” are examples of grain oils that were recovered post-distillation from a dry-grind corn ethanol process using the water refining process described herein with respect to FIG. 1 and related text. “ POET clarified” refers to the light phase (corn oil product) and “POET defoamer” refers to the heavy phase (emulsion phase). “Voila®” corn oil is also an example of a grain oil that was also recovered post-distillation from a dry-grind corn ethanol process. The “Voila®” corn oil was made using a raw starch conversion process as described in U.S. Pat. No. 7,842,484 (Lewis) and U.S. Pat. No. 7,919,291 (Lewis et al.). “3^{rd} party DCO” means third party distiller's corn oil that used a jet-cooking process to convert starch to glucose and was recovered post-distillation from a corn ethanol process and is another example of a crude vegetable oil. The Mazola® corn oil, Wesson® canola oil, and Crisco® soy oil are examples of refined vegetable oil.

As shown in FIG. 9, some oils can provide surprisingly more effective foam control when combined with hydrophobic particles such as hydrophobic silica particles, as compared to other oils (e.g., oils that have been treated to remove components such as, e.g., fatty acids), and/or as compared to mineral oil. While not being bound by theory, it is believed that one or more components that are present (endogenously present and/or chemically formed in-situ during processing of the oil) in grain oil can enhance its anti-foam characteristic. For example, free fatty acids and/or fatty acid ethyl esters can be generated during ethanol production and are present at levels not present in many food grade oils. For example, one or more processes of refining (e.g., alkali refining), deodorizing, and the like may remove free fatty acids to form refined oil, thereby reducing the anti-foam characteristic of the refined vegetable oil.

Also, while not being bound by theory, it is believed that one or more fatty acid ethyl esters present in crude vegetable oil (endogenously present and/or chemically formed in-situ during processing of the oil) may enhance its anti-foam performance as compared to other crude vegetable oils that do not have any or a lower amount of one or more endogenous fatty acid alkyl esters.

### Example 10

Example 10 shows compositional analysis of various corn oils in Table 13 below. In FIG. 10,“Voila” refers Voila® corn oil, which is a distiller's corn oil and is made in a manner similar to grain oil composition feedstock 317 in FIG. 3. “ Clarified Voila” refers to the light phase (corn oil product) and “Voila dehydrated chilled solids” refers to a dehydrated heavy phase (dehydrated emulsion phase) produced via water refining similar to that described above with respect to FIGS. 1 and 4.

TABLE 13
Compositional Analysis

| | | | | Voila | | | | |
|---|---|---|---|---|---|---|---|---|
| | | | | Dehydrated | | | | |
| | | | | Chilled | Mazola | Clarified | Crude | 3^{rd} Party |
| Test | Analyte | Unit | Voila | Solids | Corn Oil | Voila | Soy Oil | DCO |
| Ash | Ash | ppm | <1 | <1 | 16.0 | <1 | <1 | <1 |
| Brookfield Viscosity | Viscosity | cP | 55.57 | 74.60 | 60.75 | 44.90 | 45.40 | 54.03 |
| @20° C. | | | | | | | | |
| Caloric Value | Caloric Value | cal/100 g | 898.4 | 898.7 | 899.9 | 898.4 | 899.2 | 897.4 |
| Carotenes | Alpha Carotene | mcg/g | 0.2 | 0.2 | <0.200 | 0.4 | | 1.4 |
| Carotenes | Beta Carotene | mcg/g | 4.8 | 3.2 | <0.200 | 5.6 | | 16.5 |
| Carotenes | Trans Beta | mcg/g | 2.2 | 1.3 | <0.200 | 2.4 | | 7.0 |
| | Carotene | | | | | | | |
| Cloud Point | Cloud Point | ° C. | 3.2 | 6.5 | −10.0 | −10.0 | −10.2 | −10.0 |
| Color | 1″ Lovibond Red | | 12.2 | 12.0 | 3.4 | 13.1 | 3.9 | 20.0 |
| Color | 1″ Lovibond | | 70.0 | 70.0 | 50.0 | 70.0 | 70.0 | 50.0 |
| | Yellow | | | | | | | |
| Density | Density | g/cm\hat{ }3 | 0.9142 | 0.9150 | 0.9194 | 0.9150 | 0.9249 | 0.9161 |
| @20° C. | | | | | | | | |
| Elemental Analysis | Arsenic | ppm | ND | ND | ND | ND | ND | ND |
| Elemental Analysis | Cadmium | ppm | ND | ND | ND | ND | 0.5 | ND |
| Elemental Analysis | Calcium | ppm | 8.3 | 24.3 | ND | ND | 63.7 | ND |
| Elemental Analysis | Chlorides | % | 0.1 | 0.0 | 0.1 | 0.1 | 0.0 | 0.0 |
| Elemental Analysis | Copper | ppm | | 0.0 | | | | |
| Elemental Analysis | Iron | ppm | | 9.5 | | | | |
| Elemental Analysis | Lead | ppm | ND | ND | ND | ND | ND | ND |
| Elemental Analysis | Magnesium | ppm | 42.9 | 421.0 | ND | ND | 89.7 | ND |
| Elemental Analysis | Manganese | ppm | | 2.3 | | | | |
| Elemental Analysis | Mercury | ppm | ND | ND | ND | ND | ND | ND |
| Elemental Analysis | Nitrogen | ppm | ND | 0.0 | ND | ND | 0.0 | ND |
| Elemental Analysis | Phosphorus | ppm | 28.6 | 94.1 | 0.3 | 2.0 | 1240.0 | 2.4 |
| Elemental Analysis | Potassium | ppm | 23.8 | 87.3 | 4.1 | 7.2 | 718.0 | 8.7 |
| Elemental Analysis | Sodium | ppm | 71.8 | 412.0 | ND | 1.9 | ND | 1.9 |
| Elemental Analysis | Sulfur | ppm | 19.5 | 38.0 | 4.2 | 16.3 | 16.1 | 28.2 |
| Elemental Analysis | Zinc | ppm | | 0.0 | | | | |
| Fatty Acid | Ethyl Linoleate | mass | 5.41 | 4.95 | 0 | 4.07 | 0 | 2.85 |
| Ethyl Esters | | % | | | | | | |
| Fatty Acid | Ethyl Linolenate | mass | 0.08 | 0.09 | 0 | 0.06 | 0 | 0.03 |
| Ethyl Esters | | % | | | | | | |
| Fatty Acid | Ethyl Oleate | mass | 1.92 | 1.68 | 0 | 1.26 | 0 | 0.67 |
| Ethyl Esters | | % | | | | | | |
| Fatty Acid | Ethyl Palmitate | mass | 3.05 | 2.73 | 0 | 2.38 | 0 | 1.13 |
| Ethyl Esters | | % | | | | | | |
| Fatty Acid | Ethyl Stearate | mass | 0.15 | 0.13 | 0 | 0.08 | 0 | 0 |
| Ethyl Esters | | % | | | | | | |
| Fatty Acid | Total Fatty Acid | mass | 10.62 | 9.59 | 0 | 7.84 | 0 | 4.68 |
| Ethyl Esters | Ethyl Esters | % | | | | | | |
| Fatty Acid Profile | Arachidic | mg/g | 4.0 | 4.0 | 3.9 | 4.1 | | 3.6 |
| Fatty Acid Profile | Behenic | mg/g | 1.9 | 2.1 | 1.4 | 1.6 | 1.7 | |
| Fatty Acid Profile | Capric | mg/g | 0.0 | 0.0 | 0.0 | 0.0 | 0.0 | |
| Fatty Acid Profile | Caproic | mg/g | 0.0 | 0.0 | 0.0 | 0.0 | 0.0 | |
| Fatty Acid Profile | Caprylic | mg/g | 0.0 | 0.0 | 0.0 | 0.0 | 0.0 | |
| Fatty Acid Profile | Eicosenoic | mg/g | 2.7 | 2.7 | 2.7 | 2.6 | 2.6 | |
| Fatty Acid Profile | Erucic | mg/g | 0.7 | 0.7 | 0.4 | 0.5 | 0.4 | |
| Fatty Acid Profile | Lauric | mg/g | 0.1 | 0.1 | 0.0 | 0.1 | 0.0 | |
| Fatty Acid Profile | Lauroleic | mg/g | 0.0 | 0.0 | 0.0 | 0.0 | 0.0 | |
| Fatty Acid Profile | Lignoceric | mg/g | 2.1 | 2.5 | 1.6 | 2.1 | 2.2 | |
| Fatty Acid Profile | Linoleic + | mg/g | 539.2 | 532.6 | 553.4 | 541.6 | 551.2 | |
| | Isomers | | | | | | | |
| Fatty Acid Profile | Linolenic, | mg/g | 0.0 | 0.0 | 0.0 | 0.0 | 0.0 | |
| | gamma | | | | | | | |
| Fatty Acid Profile | Linolenic, alpha | mg/g | 13.5 | 13.4 | 8.9 | 13.5 | 13.6 | |
| Fatty Acid Profile | Margaric | mg/g | 0.7 | 0.7 | 0.7 | 0.7 | 0.7 | |
| Fatty Acid Profile | Margaroleic | mg/g | 0.3 | 0.3 | 0.3 | 0.3 | 0.3 | |
| Fatty Acid Profile | Myristic | mg/g | 0.6 | 0.6 | 0.3 | 0.6 | 0.4 | |
| Fatty Acid Profile | Myristoleic | mg/g | 0.0 | 0.0 | 0.0 | 0.0 | 0.0 | |
| Fatty Acid Profile | Oleic + Isomers | mg/g | 277.5 | 279.1 | 290.9 | 277.4 | 266.9 | |
| Fatty Acid Profile | Other Fatty Acids | mg/g | 0.2 | 0.3 | 0.1 | 0.1 | 0.1 | |
| Fatty Acid Profile | other isomers | mg/g | 0.0 | 0.0 | 0.0 | 0.0 | | 0.0 |
| Fatty Acid Profile | Palmitic | mg/g | 135.0 | 138.5 | 115.3 | 133.1 | | 136.0 |
| Fatty Acid Profile | Palmitoleic] | mg/g | 1.2 | 1.2 | 0.9 | 1.2 | | 1.0 |
| Fatty Acid Profile | Pentadadecylic | mg/g | 0.0 | 0.0 | 0.0 | 0.0 | | 0.0 |
| Fatty Acid Profile | Pentadadecyloleic | mg/g | 0.0 | 0.0 | 0.0 | 0.0 | | 0.0 |
| Fatty Acid Profile | Stearic | mg/g | 19.5 | 20.3 | 16.6 | 19.4 | | 18.1 |
| Fatty Acid Profile | Stearidonic | mg/g | 0.0 | 0.0 | 0.0 | 0.0 | | 0.0 |
| Fatty Acid Profile | Tetracosenoic | mg/g | 0.8 | 0.9 | 2.6 | 1.1 | | 1.2 |
| Flash Point | Flash Point | ° F. | 420.0 | 407.0 | >550 | 430.0 | 575.0 | 417.0 |
| Free Fatty Acid | Free Fatty Acid | % | 4.0 | 14.8 | 0.1 | 3.9 | 0.5 | 12.7 |
| | (Oleic) | | | | | | | |
| Free Glycerol | Glycerol | % | <0.05 | 0.1 | <0.05 | <0.05 | <0.05 | 0.1 |
| Glycerides | Diglycerides | % | 5.49 | 14.9 | 3.2 | 5.3 | 2.1 | 13.0 |
| Glycerides | Monoglycerides | % | 0.64 | 5.3 | 0.8 | 0.7 | 3.4 | 2.3 |
| Glycerides | Triglycerides | % | 82.06 | 78.3 | 86.7 | 81.1 | 86.0 | 75.5 |
| Insoluble Impurities | Insolubles | % | 0.0 | 0.1 | 0.0 | 0.0 | 0.0 | 0.0 |
| Iodine Value | Iodine | | 119.7 | 118.8 | 123.2 | 121.8 | 133.3 | 120.6 |
| Moisture | Moisture (K/F) | % | 0.2 | 0.1 | 0.0 | 0.2 | 0.1 | 0.3 |
| Oxadative Stability Index | OSI | hours | 8.5 | 3.1 | 5.0 | 8.6 | | 9.0 |
| p-Anisidine Value | p-Anisidine | | 22.8 | 21.7 | 10.6 | 21.4 | 0.6 | 22.7 |
| | Value | | | | | | | |
| Peroxide Value | Peroxide | meq/kg | 0.2 | 2.5 | 28.5 | 0.0 | 0.0 | 0.3 |
| Pour Point | Pour Point | ° C. | −6.8 | 2.3 | −8.1 | −9.3 | −6.8 | −10.6 |
| Smoke Point | Smoke Point | ° F. | 360.0 | 313.0 | 493.0 | 320.0 | 460.0 | 300.0 |
| Soaps | Soap as Sodium | ppm | 1717.0 | 7983.0 | <1 | <1 | 438.0 | <1 |
| | Oleate | | | | | | | |
| Sterols | 24-methylene- | % | 0.8 | 0.6 | 0.9 | 0.7 | 0.2 | 0.4 |
| | cholesterol | | | | | | | |
| Sterols | Apparent β- | | 69.7 | 71.0 | 71.5 | 69.5 | 55.6 | 72.2 |
| | Sitosterol | | | | | | | |
| Sterols | Brassicaserol | % | 0.1 | 0.0 | 0.0 | 0.0 | 0.1 | 0.0 |
| Sterols | Campestanol | % | 5.4 | 5.9 | 1.3 | 6.0 | 0.5 | 5.2 |
| Sterols | Campesterol | % | 16.7 | 15.4 | 18.5 | 16.7 | 23.7 | 15.2 |
| Sterols | Cholesterol | % | 0.3 | 0.7 | 0.3 | 0.2 | 0.3 | 0.1 |
| Sterols | Clerosterol | % | 0.9 | 0.7 | 0.7 | 0.9 | 0.3 | 0.8 |
| Sterols | Erythrodiol | % | 0.0 | 0.0 | 0.0 | 0.0 | 0.0 | 0.0 |
| Sterols | Sitostanol | % | 14.6 | 5.4 | 3.5 | 3.4 | 0.0 | 16.2 |
| Sterols | Stigmasterol | % | 4.7 | 4.3 | 6.2 | 4.5 | 16.2 | 4.6 |
| Sterols | Total Sterols | % | 1.9 | 1.9 | 1.1 | 1.9 | 0.5 | 2.4 |
| Sterols | Total Sterols | ppm | 19145.0 | 18763.0 | 11293.0 | 19339.0 | 4589.0 | 23653.0 |
| Sterols | Uvaol | % | 0.0 | 0.0 | 0.0 | 0.0 | 0.0 | 0.0 |
| Sterols | β-sitosterol | % | 50.0 | 49.3 | 64.4 | 48.7 | 0.7 | 50.7 |
| Sterols | Δ-5,23- | % | 0.5 | 0.9 | 0.2 | 0.4 | 0.4 | 0.4 |
| | stigmastadienol | | | | | | | |
| Sterols | Δ5,24- | % | 0.6 | 0.3 | 0.4 | 0.5 | 0.8 | 0.6 |
| | stigmasadienol | | | | | | | |
| Sterols | Δ-5-avenasterol | % | 3.2 | 14.4 | 2.4 | 15.5 | 2.2 | 3.5 |
| Sterols | Δ-7-Avenasterol | % | 1.1 | 1.0 | 0.6 | 1.2 | 1.3 | 1.2 |
| Sterols | Δ-7-Campesterol | % | 0.4 | 0.3 | 0.3 | 0.3 | 0.6 | 0.3 |
| Sterols | Δ-7-Stigmastenol | % | 1.0 | 0.7 | 0.4 | 1.0 | 1.5 | 0.9 |
| Tocopherol/Tocotrienols | Total | ppm | 491.5 | 419.5 | 560.8 | 610.4 | 1008.2 | 884.4 |
| | Tocopherols | | | | | | | |
| Tocopherol/Tocotrienols | Total | ppm | 436.1 | 435.5 | ND | 361.3 | ND | 536.7 |
| Tocopherol/Tocotrienols | Total Vitamin E | ppm | 927.6 | 855.0 | 560.8 | 971.7 | 1008.2 | 1421.1 |
| Tocopherol/Tocotrienols | α-tocopherol | ppm | 140.8 | 109.6 | 170.5 | 175.2 | 104.1 | 274.6 |
| Tocopherol/Tocotrienols | α-Tocotrienol | ppm | 151.4 | 217.6 | ND | 169.5 | ND | 201.8 |
| Tocopherol/Tocotrienols | β-tocopherol | ppm | ND | ND | ND | ND | 23.5 | 292.5 |
| Tocopherol/Tocotrienols | β-Tocotrienol | ppm | ND | ND | ND | ND | ND | ND |
| Tocopherol/Tocotrienols | γ-tocopherol | ppm | 350.7 | 275.4 | 330.8 | 394.3 | 495.7 | 277.0 |
| Tocopherol/Tocotrienols | γ-Tocotrienol | ppm | 284.7 | 217.9 | ND | 191.9 | ND | 334.9 |
| Tocopherol/Tocotrienols | δ-tocopherol | ppm | ND | 34.5 | 59.4 | 40.9 | 384.9 | 40.5 |
| Tocopherol/Tocotrienols | δ-Tocotrienol | ppm | ND | ND | ND | ND | ND | ND |
| Trans Fat | Trans Fat | % | 0.1 | 0.1 | 0.2 | 0.1 | 0.0 | 0.1 |
| Unsaponifiables Matters | Unsaponifiables | % | 1.9 | 1.7 | 0.9 | 2.0 | 0.5 | 2.1 |

The corn oil used in Examples 11-13 was first degummed via water degumming (20 wt. % water based on total oil/water mixture), and filtered with a 2 micron filter.

### Example 11—Batch Testing of Bleaching Corn Oil

This example evaluated bleaching corn oil by heating and contacting with air as an oxidizing gas. An apparatus similar to that shown in FIG. 10 was used for bleaching the oil included a 1 liter round bottom 3 neck flask with a gas sparger, temperature probe, and a septa used for sampling with a 6″ needle. 800 mL of oil was sparged with approximately 1 L/min of compressed air during the heating process. The oil was heated to 120, 130, 140, 150, and 160° C. and 15 ml samples were taken every ˜30 minutes after 2 hours of exposure to compressed air. The results indicate that relatively higher temperatures resulted in an increased rate of bleaching. It was noted that high temperatures result in slightly darker final color.

### Example 12—Continuous

This example evaluated a continuous bleaching process using a system similar to that shown in FIG. 11. An apparatus was setup to evaluate this using a hot plate, 1 liter beaker and a dual head peristaltic pump. The pump was set to feed oil and remove oil at the same rate. Vessel 1110 was stirred with a stir bar and air was sparged with 1 L/min of air. Vessel 1110 was heated to 120° C. with air flow and then the pump was turned on at 6 ml/min of flow in and out. The bleaching process was not able to keep up at this flow and the outlet became progressively darker and darker. The flow was decreased to 2 ml/min and the resulting oil was much lighter but still darker than achievable from the batch process.

### Example 13—Continuous with Sequential Temperature Bleaching

Example 13 evaluated a 2 stage bleaching process using a system similar to that shown in FIG. 12 with a first stage having a relatively higher temperature and shorter residence time and a second stage having a relatively lower temperature and longer residence time. The system for Example 13 used 1 L beakers on a hot plate. 1 L/min of air was delivered to both beaker 1211 and beaker 1212 in FIG. 12 through a glass sparger. Oil was pumped from vessel 1208 to vessel 1211, from vessel 1211 to vessel 1212, and from vessel 1212 to vessel 1213 using peristaltic pumps set at the same setting to balance the flow rates. Beaker 1211 was heated to 155±5° C. and beaker 1212 was heated to 125±5° C. After everything was up to temp the pumps were started to provide a flow through the system of 10 ml/min and 400 ml of oil was collected in beaker 1213. The pump was then turned up through a progression of flow rates (10, 15, 20, 25, 30 ml/min) and 400 ml of the resulting oil was collected for each. The residence times of vessels 1211 and 1212 are shown in the Table 14 below.

TABLE 14
Residence Times

| | tank 1211 | tank 1212 | |
|---|---|---|---|
| | volume (ml) | volume (ml) | |
| flow | 300 | 900 | total |
| through | Tank 1 mean | Tank 2 mean | residence |
| rate | residence | residence | time |
| (ml/min) | time (min) | time (min) | (min) |
| 10 | 30 | 90 | 120 |
| 15 | 20 | 60 | 80 |
| 20 | 15 | 45 | 60 |
| 25 | 12 | 36 | 48 |
| 30 | 10 | 30 | 40 |

The product from this example is similar in color to the 2.0 to 2.5 hour samples produced during the batch process in Example 11.

### Example 14—Batch Testing of Bleaching Corn Oil

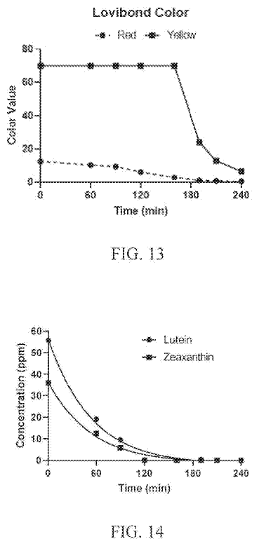

This example evaluated change in red and yellow LOVIBOND color, and change in concentration of lutein and zeoxanthin after bleaching corn oil by heating and contacting with air as an oxidizing gas. An apparatus similar to that shown in FIG. 10 was used for bleaching the oil. 800 mL of oil was sparged with approximately 1 L/min of compressed air while the oil was at 120° C. The results are shown in FIGS. 13 and 14.

It is noted that, as used in this specification and the appended claims, the singular forms “a,” “an,” and “the,” include plural referents unless expressly and unequivocally limited to one referent. Thus, for example, reference to “an antioxidant” includes two or more different antioxidants. As used herein, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is to be understood that each component, compound, substituent or parameter disclosed herein is to be interpreted as being disclosed for use alone or in combination with one or more of each and every other component, compound, substituent or parameter disclosed herein.

It is further understood that each range disclosed herein is to be interpreted as a disclosure of each specific value within the disclosed range that has the same number of significant digits. Thus, for example, a range from 1 to 4 is to be interpreted as an express disclosure of the values 1, 2, 3 and 4 as well as any range of such values.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range. That is, it is also further understood that any range between the endpoint values within the broad range is also discussed herein. Thus, a range from 1 to 4 also means a range from 1 to 3, 1 to 2, 2 to 4, 2 to 3, and so forth.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

What is claimed is:

1. A method of reducing the free fatty acid content in a grain oil, wherein the method comprises:
a) providing a first composition comprising a first grain oil portion, wherein the first composition has a first free fatty acid content based on the total weight of the first grain oil portion of the first composition;
b) exposing the first grain oil portion to an alcohol component and an esterase enzyme component to esterify at least a portion of the first free fatty acid content and form a second composition comprising a second grain oil portion, wherein the second composition has a second free fatty acid content based on the total weight of the second grain oil portion of the second composition, wherein the second free fatty acid content is less than the first free fatty acid content, wherein the esterase enzyme component is present in an amount from greater than 0 to 0.05% w/w of the weight of the first grain oil portion; and
c) separating the second composition or a composition derived from the second composition into an oil fraction and an aqueous fraction, wherein the second composition or the composition derived from the second composition has a first minerals content from 30 to 5,000 ppm, and the oil fraction has a second minerals content from 20 to 200 ppm, wherein the second minerals content is less than the first minerals content.

2. The method of claim 1, wherein the esterase enzyme component is present in an amount from greater than 0 to 0.02% w/w of the weight of the first grain oil portion.

3. The method of claim 1, wherein the first free fatty acid content is from 2 to 30% w/w based on the total weight of the first grain oil portion of the first composition and the second free fatty acid content is 10% or less w/w based on the total weight of the second grain oil portion of the second composition.

4. The method of claim 1, wherein the first composition has a first fatty acid ester alkyl content based on the total weight of the first grain oil portion of the first composition, wherein the second composition has a second fatty acid alkyl ester content based on the total weight of the second grain oil portion of the second composition, wherein the second fatty acid alkyl ester content is greater than the first fatty acid alkyl ester content.

5. The method of claim 4, wherein the first fatty acid alkyl ester content is 20% or less based on the total weight of the first grain oil portion of the first composition, and wherein the second fatty acid alkyl ester content is from 5% or greater based on the total weight of the second grain oil portion of the second composition, wherein the first fatty acid alkyl ester content is less than the second fatty acid alkyl ester content.

6. The method of claim 1, wherein the second composition or the composition derived from the second composition is thin stillage, and further comprising:
a) optionally evaporating at least a portion of water from the thin stillage to condense the thin stillage into a syrup; and
b) separating the thin stillage or syrup into the oil fraction and the aqueous fraction.

7. The method of claim 1, wherein the oil fraction is a second oil fraction, wherein the aqueous fraction is a second aqueous fraction, and wherein the second composition or the composition derived from the second composition is provided by a method comprising:
a) providing thin stillage;
b) optionally evaporating at least a portion of water from the thin stillage to condense the thin stillage into a syrup;
c) separating the thin stillage or syrup into a first oil fraction and a first aqueous fraction, wherein the first oil fraction is an emulsion; and
d) breaking the emulsion to separate the first oil fraction into the second oil fraction and the second aqueous fraction, and
e) separating the second oil fraction from the second aqueous fraction.

8. The method of claim 7, wherein breaking the emulsion comprises exposing the emulsion to an alkali base to raise the pH of the emulsion from 6 or less to 7 or greater.

9. The method of claim 7, wherein the second oil fraction is a grain oil composition feedstock and further comprising combining the grain oil composition feedstock with water to form an oil-water mixture having water in an amount of 5-50% based on the total volume of the oil-water mixture (v/v);
a) exposing the oil-water mixture to a temperature in the range from 0° C. to 50° C. for a time period at least until the oil-water mixture forms at least an oil phase and an emulsion phase; and
b) recovering at least a portion of the oil phase from the emulsion phase to form a grain oil product, wherein the grain oil product has a minerals content from 0 to less than 20 ppm.

10. The method of claim 9, further comprising bleaching the grain oil product.

11. The method of claim 10, wherein bleaching comprises contacting the grain oil product with an oxidizing gas under conditions to form a bleached grain oil product.

12. The method of claim 11, wherein the grain oil product has a color value chosen from a red color value greater than 15 on the LOVIBOND RYBN color scale according to AOCS Cc 13J-97, a yellow color value greater than 70 on the LOVIBOND RYBN color scale according to AOCS Cc 13J-97, and combinations thereof, and wherein the bleached grain oil product has a color value chosen from a red color value less than 5 on the LOVIBOND RYBN color scale according to AOCS Cc 13J-97, a yellow color value less than 30 on the LOVIBOND RYBN color scale according to AOCS Cc 13J-97, and combinations thereof

13. The method of claim 11, wherein the bleached grain oil product has a Brookfield viscosity in the range from 20 to less than 40 centiPoise (cP) when measured at 40° C. and #SC 418 spindle.

14. A method of bleaching a grain oil product to provide a bleached grain oil product, wherein the method comprises:
a) providing a grain oil product comprising one or more pigments, wherein the grain oil product has an oxidative stability value of 7 hours or less according to AOCS Official Method Cd 12b-92-Reapproved 1997; and
b) contacting the grain oil product with an oxidizing gas under conditions to form a bleached grain oil product.

15. The method of claim 14, wherein contacting the grain oil product with an oxidizing gas under conditions to form a bleached grain oil product comprises contacting the grain oil product with an oxidizing gas for a time period from 30 minutes to 6 hours while the grain oil product is at a temperature from 100° C. to 180° C.

16. The method of claim 14, wherein contacting the grain oil product with an oxidizing gas is a continuous process that comprises causing the grain oil product to flow through at least two vessels to form a bleached grain oil product.

17. The method of claim 16, where the continuous process comprises:
a) causing the grain oil product to flow through a first vessel to form a partially bleached grain oil product, wherein the grain oil product is exposed to a temperature from 150° C. to 170° C. in the first vessel, and wherein the grain oil product has a residence time from 30 to 90 minutes in the first vessel; and
b) causing the partially bleached grain oil product to flow through a second vessel to form a bleached grain oil product, wherein the partially bleached grain oil product is exposed to a temperature from 110° C. to 130° C. in the second vessel, and wherein the partially bleached grain oil product has a residence time from 1.5 to 2.5 hours in the second vessel.

18. The method of claim 14, wherein the grain oil product is filtered by passing the grain oil product through a 3 micron or less filter prior to contacting the grain oil product with an oxidizing gas under conditions to form the bleached grain oil product.

19. The method of claim 14, wherein the bleached grain oil product has a Brookfield viscosity in the range from 20 to less than 40 centiPoise (cP) when measured at 40° C. and #SC 4-18 spindle.

20. The method of claim 14, further comprising providing the grain oil product, wherein providing comprises:
a) combining a grain oil composition feedstock with water to form an oil-water mixture having water in an amount of 5-50% based on the total volume of the oil-water mixture (v/v);
b) exposing the oil-water mixture to a temperature in the range from 0° C. to 50° C. for a time period at least until the oil-water mixture forms at least an oil phase and an emulsion phase;
c) recovering at least a portion of the oil phase from the emulsion phase to form the grain oil product, wherein the grain oil product has a minerals content from 0 to 500 ppm.
